(19) United States
(12) Patent Application Publication
(10) Pub. No.: US 2005/0079200 A1
(43) Pub. Date: Apr. 14, 2005

(54) Biocompatibly coated medical implants

(76) Inventors: Jorg Rathenow, Wiesbaden (DE); Andreas Ban, Koblenz (DE); Jurgen Kunstmann, Bad Soden (DE); Bernhard Mayer, Mainz (DE); Soheil Asgari, Munchen (DE)

Correspondence Address: FROMMER LAWRENCE & HAUG, 745 FIFTH AVENUE- 10TH FL., NEW YORK, NY, 10151, US

(21) Appl. No.: 10/938,995

(22) Filed: Sep. 10, 2004

Related U.S. Application Data
(63) Continuation-in-part of application No. PCT/EP04/04985, filed on May 10, 2004.

(30) Foreign Application Priority Data
May 16, 2003 (DE) 103 22 182.4
May 28, 2003 (DE) 103 24 415.8
Jul. 21, 2003 (DE) 103 33 098.4

(51) Int. Cl. A61F 2/02; B05D 3/00; A61F 2/06; A61F 2/28; A61F 2/24

(52) U.S. Cl. 424/423; 427/2.24; 623/23.5; 623/1.46; 623/2.42

(57) ABSTRACT

Implantable medical devices with biocompatible coatings and processes for their production are described. The present invention relates in particular to medical implantable devices coated with a carbon-containing layer which devices are produced by at least partially coating the device with a polymer film and heating the polymer film in an atmosphere which is essentially free from oxygen to temperatures in the region of 200° C. to 2500° C., a carbon-containing layer being produced on the implantable medical device.

### INCORPORATION BY REFERENCE

This application is a continuation-in-part application of international patent application Serial No. PCT/EP2004/004985 filed May 10, 2004, which claims benefit of German patent application Serial Nos. DE 103 22 182.4 filed May 16, 2003; DE 103 24 415.8 filed May 28, 2003 and DE 103 33 098.4 filed Jul. 21, 2003.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relates to implantable medical devices with biocompatible coatings and a process for their production. In particular, the present invention relates to medical implantable devices coated with a carbon-containing layer which devices can be obtained by at least partial coating of the device with a polymer film and heating of the polymer film to temperatures in the region of 200° C. to 2500° C. in an atmosphere which is essentially free from oxygen, a carbon-containing layer being produced on the implantable medical device.

### BACKGROUND OF THE INVENTION

Medical implants such as surgical and/or orthopaedic screws, plates, arthroplasties, artificial heart valves, vascular prostheses, stents as well as subcutaneous or intramuscular implantable depots of active principles are produced from a wide variety of materials which are selected according to the specific biochemical and mechanical properties concerned. These materials must be suitable for permanent use in the body, not release toxic substances and must exhibit certain mechanical and biochemical properties.

However, the metals or metal alloys as well as ceramic materials frequently used for stents and arthroplasties, for example, frequently exhibit disadvantages regarding their biocompatibility, particularly during permanent use. As a result of chemical and/or physical irritation, implants cause inflammatory tissue and immune responses, among other things, such that incompatibility reactions occur in the sense of chronic inflammation reactions with defence and rejection responses, excessive scarring or tissue degradation which, in extreme cases, necessarily lead to the implant having to be removed and replaced or additional therapeutic interventions of an invasive or non-invasive nature being indicated.

Lack of compatibility with the surrounding tissue in the case of coronary stents, for example, leads to high rates of restenosis since, on the one hand, the intima of the vascular wall has a tendency towards inflammation-induced macrophages reaction with scarring and, on the other hand, both the direct surface properties and the pathologically changed vascular wall in the area of the stent lead to aggregation of thrombocytes at the vascular implant itself and on vascular walls which have changed in an inflammatory manner. Both mechanisms provide support to a reciprocally influencing inflammation and incompatibility process which leads in 20-30% of the patients provided with stents by intervention to a renewed narrowing of the coronary artery requiring treatment.

For this reasons, various approaches have been made in the prior art for coating the surfaces of medical implants in a suitable manner in order to increase the biocompatibility of the materials used and to prevent defence and/or rejection reactions.

In U.S. Pat. No. 5,891,507, for example, processes for coating the surface of metal stents with silicone, polytetrafluoroethylene and biological materials such as heparin or growth factors are described which increase the biocompatibility of the metal stent.

Apart from polymer layers, layers based on carbon have proved to be particularly advantageous.

From DE 199 51 477, for example, coronary stents with a coating of amorphous silicon carbide are thus known which increase the biocompatibility of the stent material. U.S. Pat. No. 6,569,107 describes carbon-coated stents in the case of which the carbon material has been applied by chemical or physical vapour phase deposition methods (CVD or PVD). In U.S. Pat. No. 5,163,958, too, tubular endoprostheses or stents with a carbon-coated surface are described which possesses antithrombogenic properties. WO 02/09791 describes intravascular stents with coatings produced by CVD of siloxanes.

The deposition of pyrolytic carbon under PVD or CVD conditions requires the careful selection of suitable gaseous or vaporisable carbon precursors which are then deposited on the implant frequently at high temperatures, in some cases under plasma conditions, in an inert gas or high vacuum atmosphere.

Apart from the CVD methods for depositing carbon, different sputter processes operating in a high vacuum are described in the prior art for the production of pyrolytic carbon layers with different structures; compare in this respect e.g. U.S. Pat. No. 6,355,350.

All these processes of the prior art possess the joint feature that the deposition of carbon substrates takes place partly under extreme temperature and/or pressure conditions and by using complex process controls.

A further disadvantage of the processes of the prior art is that, as a result of different thermal expansion efficiencies of materials from which the implants are made and the CDV layers applied, only a low level of adhesion of the layer is frequently achieved on the implant as a result of which detachment, cracks and a deterioration of the surface quality occur having a negative effect on the usefulness of the implants.

Consequently, there is a requirement for cost-effective processes simple to use for coating implantable medical devices with a carbon-based material which processes are capable of providing biocompatible surface coatings of carbon-containing material.

Moreover, there is a requirement for cost-effectively producible biocompatibly coated medical implants with improved properties.

Citation or identification of any document in this application is not an admission that such document is available as prior art to the present invention.

### SUMMARY OF THE INVENTION

It is a task of the present invention to provide a process for the production of biocompatible coatings on implantable medical devices which process manages with using starting materials which are cost-effective and have properties variable in many ways and which uses processing conditions simple to control.

It is a further task of the present invention to provide implantable medical devices equipped with carbon-containing coatings which devices exhibit an increased biocompatibility.

It is a further task of the present invention to provide biocompatibly coated medical implants whose coating allows the application of medical active principles onto or into the surface of the implant.

It is also a further task of the present invention to provide coated medical implants which are capable of liberating in a targeted and, if necessary, controlled manner applied pharmacologically effective substances after insertion of the implant into the human body.

It is a further task of the present invention to provide implantable active principle depots with a coating capable of controlling the release of active principles from the depot.

The solution, according to the invention, of the above-mentioned tasks consists of a process and coated medical implants obtainable therewith as defined in the independent claims. Preferred embodiments of the process according to the invention and/or the products according to the invention result from the dependent sub-claims.

Within the framework of the present invention it has been found that carbon-containing layers can be produced on implantable medical devices of widely differing types in a simple and reproducible manner by coating the device initially at least partially with a polymer film which is subsequently carbonised and/or pyrolysed in an essentially oxygen-free atmosphere at high temperatures. Preferably, the resulting carbon-containing layer(s) are subsequently loaded with active principles, microorganisms or living cells. Also, it is possible, as an alternative or additionally, to coat at least partially with biodegradable and/or resorbable polymers or non-biodegradable and/or resorbable polymers.

Accordingly, the process according to the invention for the production of biocompatible coatings on implantable medical devices comprises the following steps:
a) at least partial coating of the medical device with a polymer film by means of a suitable coating and/or application process;
b) heating of the polymer film in an atmosphere which is essentially free from oxygen to temperatures in the region of 200° C. to 2500° C., for the production of a carbon-containing layer on the medical device.

Within the framework of the present invention, carbonising or pyrolysis is understood to mean the partial thermal decomposition or coking of carbon-containing starting compounds which, as a rule, consist of oligo or polymer materials based on hydrocarbons which, following carbonisation, leave behind carbon-containing layers as a function of the temperature and pressure conditions selected and the type of polymer materials used, which layers can be adjusted accurately regarding their structure within the range of amorphous to highly ordered crystalline graphite-type structures and regarding their porosity and surface properties.

The process according to the invention can be used not only for coating implantable medical devices but, in its most general aspect, also in general for the production of carbon-containing coatings on substrates of any desired type. The statements made in the following regarding implants as a substrate consequently apply without exception also to other substrates for other purposes.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DETAILED DESCRIPTION

By means of the process according to the invention, biocompatible, carbon-containing coatings can be applied onto implantable medical devices.

The terms “implantable, medical device” and “implant” will be used synonymously in the following and comprise medical or therapeutic implants such as e.g. vascular endoprostheses, intraluminal endoprostheses, stents, coronary stents, peripheral stents, surgical and/or orthopaedic implants for temporary purposes such as surgical screws, plates, pins and other fixing facilities, permanent surgical or orthopaedic implants such as bone prostheses or arthroplasties, e.g. artificial hip joints or knee joints, joint cavity inserts, screws, plates, pins, implantable orthopaedic fixing aids, vertebral body replacements as well as artificial hearts and parts thereof, artificial heart valves, cardiac pacemaker housings, electrodes, subcutaneously and/or intramuscularly insertible implants, active principle depots and microchips and such like.

The implants that can be coated in a biocompatible manner by means of the process of the present invention may consist of almost any desired, preferably essentially temperature-stable materials, in particular of all materials from which implants are made.

Examples in this respect are amorphous and/or (partially) crystalline carbon, complete carbon material, porous carbon, graphite, composite carbon materials, carbon fibres, ceramics such as e.g. zeolites, silicates, aluminium oxides, aluminosilicates, silicon carbide, silicon nitride; metal carbides, metal oxides, metal nitrides, metal carbonitrides, metal oxycarbides, metal oxynitrides and metal oxycarbonitrides of the transition metals such as titanium, zirconium, hafnium, vanadium, niobium, tantalum, chromium, molybdenum, tungsten, manganese, rhenium, iron, cobalt, nickel; metals and metal alloys, in particular the noble metals gold, silver, ruthenium, rhodium, palladium, osmium, iridium, platinum; metals and metal alloys of titanium, zircon, hafnium, vanadium, niobium, tantalum, chromium, molybdenum, tungsten, manganese, rhenium, iron, cobalt, nickel, copper; steel, in particular stainless steel, shape memory alloys such as nitinol, nickel-titanium alloys, glass, stone, glass fibres, minerals, natural or synthetic bone substance bone, imitates based on alkaline earth metal carbonates such as calcium carbonate, magnesium carbonate, strontium carbonate and any desired combinations of the above-mentioned materials.

In addition, materials can also be coated which are first converted into their final form under the carbonising conditions. Examples in this respect are moulded bodies of paper, fibre materials and polymeric materials which, after coating with the polymer film, are converted together with the latter into coated carbon implants.

In the process according to the invention, the manufacture of coated implants is also possible starting out in principle from ceramic preliminary stages of the implant such as e.g. green ceramic bodies which, after coating with the polymer film, can be cured or sintered into their final application form in combination with carbonising of the polymer film. In this way, it is possible to use e.g. commercial and/or convention ceramics (boron nitride, silicon carbide etc.) or nanocrystalline green bodies of zirconium oxide and alpha Al₂O₃ or gamma Al₂O₃, or compressed amorphous nanoscale ALOOH aerogel leading to nanoporous carbon-coated moulded bodies at temperatures of approximately 500-2000°, preferably, however approximately 800° C., coatings with porosities of approximately 10-100 nm being obtainable. Preferred fields of application in this respect are e.g. full implants for the reconstruction of joints which have an improved biocompatibility and lead to a homogeneous layer composite.

The process according to the invention solves the problem of delamination of coated ceramic implants which, when subjected to biomechanical torsion, tension and elongation strains, usually have a tendency towards the abrasion of coatings applied secondarily.

The coatable, implantable medical devices according to the invention can have almost any desired external shape; the process according to the invention is not restricted to certain structures. According to the process of the invention, the implants can be coated entirely or partially with a polymer film which is subsequently carbonised to form a carbon-containing layer.

In preferred embodiments of the present invention, the medical implants to be coated comprise stents, in particular medical stents. Using the process according to the invention, it is possible to apply, in just as simple and advantageous a manner, surface coatings based on carbon and/or containing carbon onto stents of stainless steel, platinum-containing radiopaque steel alloys, the so-called PERSS (platinum enhanced radiopaque stainless steel alloys), cobalt alloys, titanium alloys, high melting alloys e.g. based on niobium, tantalum, tungsten and molybdenum, noble metal alloys, nitinol alloys as well as magnesium alloys and mixtures of the above-mentioned substances.

Preferred implants within the framework of the present invention are stents of stainless steel, in particular Fe-18Cr-14Ni-2.5Mo (“316LVM” ASTM F138), Fe-21Cr-10Ni-3.5Mn-2.5Mo (ASTM F 1586), Fe-22Cr-13Ni-5Mn (ASTM F 1314), Fe-23Mn-21Cr-1Mo-1N (nickel-free stainless steel); of cobalt alloys such as e.g. Co-20Cr-15W-10Ni (“L605” ASTM F90), Co-20Cr-35Ni-10Mo (“MP35N” ASTM F 562), Co-20Cr-16Ni-16Fe-7Mo (“Phynox” ASTM F 1058); examples of preferred titanium alloys are CP titanium (ASTM F 67, grade 1), Ti-6A1-4V (alpha/beta ASTM F 136), Ti-6A1-7Nb (alpha/beta ASTM F1295), Ti-15Mo (beta grade ASTM F2066); stents of noble metal alloys, in particular iridium-containing alloys such as Pt-10Ir; nitinol alloys such as martensitic, superelastic and cold worked (preferably 40%) nitinols and magnesium alloys such as Mg-3A1-1Z.

According to the process of the invention, the implants are coated with one or several layers of polymer film at least partially on one of their external surfaces, in preferred applications on their entire external surface.

In one embodiment of the invention, the polymer film can be present in the form of a polymer sheeting which can be applied and/or bonded onto the implant by suitable processes, e.g. by sheet shrinking methods. Thermoplastic polymer sheeting can be applied to essentially adhere firmly on most substrates, in particular also in the heated state.

Moreover, the polymer film may also comprise a coating of the implant with varnishes, polymeric or partially polymeric coatings, immersion coatings, spray coatings or coatings of polymer solutions or polymer suspensions as well as polymer layers applied by lamination.

Preferred coatings can be obtained by the surface parylenation of the substrates. In this case, the substrates are treated with paracyclophane initially at an elevated temperature, usually approximately 600° C., whereupon a polymer film of poly (p-xylylene) is formed on the surface of the substrates. This film can be converted into carbon in a subsequent carbonising and/or pyrolysis step.

In particularly preferred embodiments, the sequence of the steps of parylenation and carbonising is repeated several times.

Further preferred embodiments of polymer films consist of polymer foam systems e.g. phenolic foams, polyolefin foams, polystyrene foams, polyurethane foams, fluoropolymer foams which can be converted into porous carbon layers in a subsequent carbonising and/or pyrolysis step.

For the polymer films in the form of sheeting, varnishes, polymeric coatings, immersion coatings, spray coatings or coverings as well as polymer layers applied by lamination, it is possible to use e.g. homopolymers or copolymers of aliphatic or aromatic polyolefins such as polyethylene, polypropylene, polybutene, polyisobutene, polypentene; polybutadiene; polyvinyls such as polyvinyl chloride or polyvinyl alcohol, poly(meth)acrylic acid, polyacrylocyano acrylate; polyacrylonitril, polyamide, polyester, polyurethane, polystyrene, polytetrafluoroethylene; polymers such as collagen, albumin, gelatine, hyaluronic acid, starch, celluloses such as methylcellulose, hydroxypropyl cellulose, hydroxypropyl methylcellulose, carboxymethylcellulose phthalate; waxes, paraffin waxes, Fischer-Tropsch waxes; casein, dextrans, polysaccharides, fibrinogen, poly(D,L-lactides), poly(D,L-lactide coglycolides), polyglycolides, polyhydroxybutylates, polyalkyl carbonates, polyorthoesters, polyesters, polyhydroxyvaleric acid, polydioxanones, polyethylene terephthalates, polymaleate acid, polytartronic acid, polyanhydrides, polyphosphazenes, polyamino acids; polyethylene vinyl acetate, silicones; poly(ester urethanes), poly(ether urethanes), poly(ester ureas), polyethers such as polyethylene oxide, polypropylene oxide, pluronics, polytetramethylene glycol; polyvinylpyrrolidone, poly(vinyl acetate phthalate) as well as their copolymers, mixtures and combinations of these homopolymers or copolymers.

Suitable varnish-based polymer films, e.g. films and/or coverings produced from a one-component or two-component varnish which have a binder base of alkyd resin, chlorinated rubber, epoxy resin, formaldehyde resin, (meth)acrylate resin, phenol resin, alkyl phenol resin, amine resin, melamine resin, oil base, nitro base, vinyl ester resin, Novolac® epoxy resin, polyester, polyurethane, tar, tar-like materials, tar pitch, bitumen, starch, cellulose, shellac, waxes, organic materials of renewable raw materials or combinations thereof are particularly preferred.

Varnishes based on phenol resins and/or melamine resins which optionally may be epoxidised completely or partially, e.g. commercial packaging varnish as well as one-component or two-component varnishes optionally based on epoxidised aromatic hydrocarbon resins are particularly preferred.

In the process according to the invention, several layers of the above-mentioned polymer films can be applied onto the implant which are then carbonised together. By using different polymer film materials, possibly additives in individual polymer films, or films of different thickness, it is possible to apply in this way gradient coatings in a controlled manner onto the implant e.g. with variable porosity or absorption profiles within the coatings. Moreover, the sequence of the steps of polymer film coating and carbonising can be repeated once and optionally also several times in order to obtain carbon-containing multi-layer coatings on the implant. For this purpose, the polymer films or substrates can be pre-structured or modified by means of additives. It is also possible to use suitable after-treatment steps as described in the following after each or after individual ones of the sequences of the steps of polymer film coating and carbonising of the process according to the invention, such as e.g. an oxidative treatment of individual layers.

The use of polymer films coated with the above-mentioned varnishes or coating solutions for coating the implants e.g. by laminating techniques such as thermal, pressure pressing or wet-in-wet techniques can be applied advantageously according to the invention.

In certain embodiments of the present invention, the polymer film can be equipped with additives which influence the carbonising behaviour of the film and/or the macroscopic properties of the substrate layer based on carbon resulting from the process. Examples of suitable additives are fillers, pore forming agents, metals, metal compounds, alloys and metal powders, extenders, lubricants, slip additives etc. Examples of inorganic additives or fillers are silicon oxides or aluminium oxides, aluminosilicates, zeolites, zirconium oxides, titanium oxides, talcum, graphite, carbon black, fullerines, clay materials, phyllosilicates, suicides, nitrides, metal powders, in particular of catalytically active transition metals such as copper, gold and silver, titanium, zirconium, hafnium, vanadium, niobium, tantalum, chromium, molybdenum, tungsten, manganese, rhenium, iron, cobalt, nickel, ruthenium, rhodium, palladium, osmium, iridium or platinum.

Using such additives in the polymer film, it is possible to modify and adjust e.g. biological, mechanical and thermal properties of the films and of the resulting carbon coatings. By incorporating e.g. layer silicates, nanoparticles, inorganic nanocomposites, metals, metal oxides it is thus possible to adjust the thermal expansion coefficient of the carbon layer to that of a substrate of ceramic in such a way that the carbon-based coating applied adheres firmly even in the case of strong differences in temperature. On the basis of simple routine experiments, the person skilled in the art will select a suitable combination of polymer film and additive in order to obtain the desired adhesion and expansion properties of the carbon-containing layer for each implant material. Thus, the use of aluminium-based fillers will lead to an increase in the thermal expansion coefficient and the addition of fillers based on glass, graphite or quartz will lead to a reduction in the thermal expansion coefficient such that the thermal expansion coefficient can be adjusted correspondingly individually by mixing the components in the polymer system. A further possible adjustment of the properties can take place, as an example and non-exclusively, by the preparation of a fibre composite by adding carbon fibres, polymer fibres, glass fibres or other fibres in the woven or non-woven form leading to a substantial increase in the elasticity of the coating.

The biocompatibility of the layers obtained can also be modified and additionally increased by a suitable selection of additives in the polymer film.

In a preferred embodiment of the invention, it is possible by further coating of the polymer film with epoxy resin, phenol resins, tar, tar pitch, bitumen, rubber, polychloroprene or poly(styrene cobutadiene) latex materials, waxes, siloxanes, silicates, metal salts and/or metal salt solutions, e.g. transition metal salts, carbon black, fullerines, activated carbon powder, carbon molecular sieve, perovskite, aluminium oxide, silicon oxide, silicon carbide, boron nitride, silicon nitride, noble metal powders such as e.g. Pt, Pd, Au or Ag and combinations thereof or by the targeted incorporation of such materials into the polymer film structure, to influence or refine the properties of the porous carbon-based coating obtained after the pyrolysis and/or carbonisation in a controlled manner or to produce multi-layer coatings, in particular multi-layer coatings with layers of different porosity.

During the production of coated substrates according to the invention, there is the possibility of improving the adhesion of the layer applied onto the substrate by incorporating the above-mentioned additives into the polymer film, e.g. by applying silanes, polyaniline or porous titanium layers and, if necessary, of adjusting the thermal expansion coefficient of the external layer to that of the substrate such that these coated substrates become more resistant to fractures within and to detachment of the coating. Consequently, these coatings are more durable and more stable over time during practical use than conventional products of this type.

The application or incorporation of metals and metal salts, in particular also of noble metals and transition metals, makes it possible to adjust the chemical, biological and absorptive properties of the resulting carbon-based coatings to the desired requirements such that the resulting coating can be equipped also with heterogeneous catalytic properties, for example, for special applications. In this way, it is possible by incorporating silicon salts, titanium salts, zirconium salts or tantalum salts during carbonisation to form the corresponding metal carbide phases which increase the resistance of the layer to oxidisation, among other things.

The polymer films used in the process according to the invention have the advantage that they can be produced or are commercially available in a simple manner in almost any desired dimension. Polymer sheeting and varnishes are easily available, cost effective and can be applied in a simple manner to implants of different types and form. The polymer films used according to the invention can be structured in a suitable manner before pyrolysis or carbonising by folding, embossing, stamping, printing, extruding, piling, injection moulding and such like before or after they have been applied onto the implant. In this way, certain structures of a regular or irregular type can be incorporated into the carbon coating produced by the process according to the invention.

The polymer films which can be used according to the invention and consist of coatings in the form of varnishes or coverings can be applied onto the implant from the liquid, pulpy or paste-type state, e.g. by brush coating, spreading, varnishing, doctor blade application, spin coating, dispersion or melt coating, extruding, casting, immersing, spraying, printing or also as hot melts, from the solid state by powder coating, spraying of sprayable particles, flame spray processes, sintering or such like according to methods known as such. If necessary, the polymeric material can be dissolved or suspended in suitable solvents for this purpose. The lamination of suitably formed substrates with polymer materials or sheeting suitable for this purpose is also a method that can be used according to the invention for coating the implant with a polymer film.

When coating stents with polymer films, the application of the polymer and/or a solution thereof by pressure processes as described in DE 10351150 whose disclosure is included herein in full, is particularly preferred. This process permits, in particular, a precise and reproducible adjustment of the layer thickness of the polymer material applied.

In preferred embodiments, the polymer film is applied as a liquid polymer or polymer solution in a suitable solvent or solvent mixture, if necessary with subsequent drying. Suitable solvents comprise, for example, methanol, ethanol, N-propanol, isopropanol, butoxydiglycol, butoxyethanol, butoxyisopropanol, butoxypropanol, n-butyl alcohol, t-butyl alcohol, butylene glycol, butyl octanol, diethylene glycol, dimethoxydiglycol, dimethyl ether, dipropylene glycol, ethoxydiglycol, ethoxyethanol, ethyl hexane diol, glycol, hexane diol, 1,2,6-hexane triol, hexyl alcohol, hexylene glycol, isobutoxy propanol, isopentyl diol, 3-methoxybutanol, methoxydiglycol, methoxyethanol, methoxyisopropanol, methoxymethylbutanol, methoxy PEG-10, methylal, methyl hexyl ether, methyl propane diol, neopentyl glycol, PEG-4, PEG-6, PEG-7, PEG-8, PEG-9, PEG-6-methyl ether, pentylene glycol, PPG-7, PPG-2-buteth-3, PPG-2 butyl ether, PPG-3 butyl ether, PPG-2 methyl ether, PPG-3 methyl ether, PPG-2 propyl ether, propane diol, propylene glycol, propylene glycol butyl ether, propylene glycol propyl ether, tetrahydrofuran, trimethyl hexanol, phenol, benzene, toluene, xylene; as well as water, if necessary in mixture with dispersants and mixtures of the above-named substances.

Preferred solvents comprise one or several organic solvents from the group of ethanol, isopropanol, n-propanol, dipropylene glycol methyl ether and butoxyisopropanol (1,2-propylene glycol-n-butyl ether), tetrahydrofuran, phenol, benzene, toluene, xylene, preferably ethanol, isopropanol, n-propanol and/or dipropylene glycol methyl ether, in particular isopropanol and/or n-propanol.

In preferred embodiments of the present invention, the implantable medical devices can also be coated repeatedly with several polymer films of the same polymer in the same or different film thickness or different polymers in the same or different film thickness. In this way, it is possible to combine, for example, lower lying, more porous layers with narrow-pore layers placed above them which are capable of suitably delaying the release of active principles deposited in the strongly porous layer.

As an alternative to coating of the implant with a polymer film and a subsequent carbonising step, it is also possible according to the invention to directly spray a polymer film-producing coating system, e.g. a varnish based on aromatic resins directly onto a preheated implant e.g. by means of excess pressure in order to carbonise the film layer sprayed on directly on the hot implant surface.

The polymer film applied onto the implant is dried, if necessary, and subsequently subjected to a pyrolytic decomposition under carbonisation conditions. In this case, the polymer film(s) coated onto the implant is heated, i.e. carbonised at elevated temperature in an atmosphere essentially free of oxygen. The temperature of the carbonising step is preferably in the region of 200° C. to 2500° C. and is chosen by the person skilled in the art as a function of the specific temperature-dependent properties of the polymer films and the implants used.

Preferred, generally applicable temperatures for the carbonising step of the process according to the invention are in the region of 200° C. to approximately 1200° C. In the case of some embodiments, temperatures in the region of 250° C. to 700° C. are preferred. In general, the temperature is chosen depending on the properties of the materials used in such a way that the polymer film is transformed essentially completely into carbon-containing solids with as low a temperature application as possible. By suitably selecting and/or controlling the pyrolysis temperature, the porosity, the strength and rigidity of the material as well as further properties can be adjusted in a controlled manner.

Depending on the type of polymer film used and the carbonisation conditions selected, in particular the composition of the atmosphere, the temperatures or the temperature programmes and the pressure conditions selected, it is possible to adjust and/or vary the type and structure of the carbon-containing layer deposited in a controlled manner by means of the process according to the invention. When using pure carbon-based polymer films, for example, in an oxygen-free atmosphere at temperatures of up to approximately 1000° C., a deposition of essentially amorphous carbon thus takes place whereas at temperatures above 2000° C., highly ordered crystalline graphite structures are obtained. In the region between these two temperatures, partially crystalline carbon-containing layers of different densities and porosities can be obtained.

A further example is the use of foamed polymer films, e.g. foamed polyurethanes, which allow relatively porous carbon layers with pore sizes in the lower millimetre range to be obtained during carbonisation. Through the thickness of the polymer film applied and the temperature and pressure conditions selected, it is also possible to vary, during the pyrolysis, the layer thickness of the deposited carbon-containing layer within wide limits ranging from carbon mono-layers via almost invisible layers in the nanometre range to varnish layer thicknesses of the dry layer of 10 to 40 micrometres to thicker depot layer thickness in the millimetre range to the centimetre range. The latter is preferred particularly in the case of implants of full carbon materials, in particular bone implants.

By suitably selecting the polymer film material and the carbonising conditions, depot layers resembling molecular sieve with specifically controllable pore sizes and sieve properties can thus be obtained which allow the covalent, adsorptive or absorptive or electrostatic binding of active principles or surface modifications.

Preferably, the porosity is produced in the layers according to the invention on implants by treatment processes such as described in DE 103 35 131 and PCT/EP04/00077 whose disclosures are herewith incorporated in full.

The atmosphere during the carbonising step of the process according to the invention is essentially free from oxygen, preferably has O₂ contents less than 10 ppm, particularly preferably less than 1 ppm. The use of inert gas atmospheres, e.g. nitrogen, noble metals such as argon, neon and any other inert gases or gas compounds not reacting with carbon as well as mixtures of inert gases is preferred. Nitrogen and/or argon are preferred.

Usually, the carbonisation step is carried out at normal pressure in the presence of insert gases such as those mentioned above. If necessary, however, higher inert gas pressures can advantageously be used. In some embodiments of the process according to the invention, carbonisation can also take place at reduced pressure and/or under vacuum.

The carbonisation step is preferably carried out in a batch-wise process in suitable ovens but can also be carried out in continuous oven processes which, if necessary, may be preferable. The if necessary structured, pre-treated implants coated with polymer film are passed to the oven on one side and discharged from the oven at the other end. In preferred embodiments, the implant coated with polymer film can rest in the oven on a perforated plate, a sieve or such like such that a reduced pressure can be applied through the polymer film during pyrolysis and/or carbonisation. This allows not only simple fixing of the implants in the oven but also a suction treatment and optimum flow of inert gas through the films and/or assemblies during pyrolysis and/or carbonisation.

The oven can be divided into individual segments by corresponding inert gas gates in which segments one or several pyrolysis and/or carbonisation steps can be carried out in sequence, if necessary under different pyrolysis and/or carbonisation conditions, such as different temperature stages, different inert gases and/or a vacuum, for example.

Moreover, after-treatment steps such as post-activation by reduction or oxidation or impregnation with metal salt solutions etc. can be carried out in corresponding segments of the oven, if necessary.

As an alternative, the carbonisation can also be carried out in a closed oven, this being particularly preferable if the pyrolysis and/or carbonisation is to be carried out in a vacuum.

During the pyrolysis and/or carbonisation in the process according to the invention, a decrease in the weight of the polymer film by approximately 5% to 95%, preferably approximately 40% to 90%, in particular 50% to 70%, usually takes place, depending on the starting material and the pretreatment used.

The carbon-based coating produced according to the invention on the implants and/or substrates generally has a carbon content, depending on the starting material, quantity and type of filler materials, of at least 1% by weight, preferably at least 25%, if necessary also at least 60% and particularly preferably at least 75%. Coatings particularly preferred according to the invention have a carbon content of at least 50% by weight.

In preferred embodiments of the process according to the invention, the physical and chemical properties of the carbon-based coating are further modified after pyrolysis and/or carbonisation by suitable treatment steps and adjusted to the application purpose desired in each case.

Suitable after-treatments are, for example, reducing or oxidative after-treatment steps during which the coating is treated with suitable reducing agents and/or oxidising agents such as hydrogen, carbon dioxide such as N₂O, steam, oxygen, air, nitric acid and such like as well as, if necessary, mixtures of these.

However, if necessary, the after-treatment steps can be carried out at elevated temperature, though below the pyrolysis temperature, e.g. of 40° C. to 1000° C., preferably 70° C. to 900° C., particularly preferably 100° C. to 850° C., in particular preferably 200° C. to 800° C. and in particular at approximately 700° C. In particularly preferred embodiments, the coating produced according to the invention is modified reductively or oxidatively or with a combination of these after-treatment steps at room temperature.

By oxidative and/or reductive treatment or by the incorporation of additives, fillers or functional materials, the surface properties of the coatings produced according to the invention can be influenced and/or modified in a controlled manner. For example, it is possible to render the surface properties of the coating hydrophilic or hydrophobic by incorporating inorganic nanoparticles or nanocomposites such as layer silicates.

It is also possible to provide the coatings produced according to the invention subsequently with biocompatible surfaces by incorporating suitable additives and to use them as carriers or depots of medicinal substances. For this purpose, it is possible to incorporate e.g. medicaments or enzymes into the material, it being possible for the former to be liberated, if necessary, in a controlled manner by suitable retarding and/or selective permeation properties of the coatings.

According to the process of the invention, it is also possible to suitably modify the coating on the implant, e.g. by varying the pore sizes by suitable or oxidative reductive after-treatment steps such as oxidation in the air at elevated temperatures, boiling in oxidising acids, alkalis or admixing volatile components which are degraded completely during carbonisation and leave pores behind in the carbon-containing layer.

If necessary, the carbonising layer can also be subjected in a further optional process step to a so-called CVD process (chemical vapour deposition) or a CVI process (chemical vapour infiltration) in order to further modify the surface structure or pore structure and their properties. For this purpose, the carbonised coating is treated with suitable precursor gases splitting off carbon at high temperatures. Other elements, too, can be deposited therewith, e.g. silicon. Such processes have been known in the state of the art for a long time.

Almost all known saturated and unsaturated hydrocarbons with a suitable volatility under CVD conditions are suitable for use as precursor splitting off carbon. Examples of these are methane, ethane, ethylene, acetylene, linear and branched alkanes, alkenes and alkines with carbon numbers of C₁-C₂₀, aromatic hydrocarbons such as benzene, naphthalene etc., and singly and multiply alkyl substituted, alkenyl substituted and alkinyl substituted aromatics such as toluene, xylene, cresol, styrene, parylenes etc.

As ceramic precursor, BCI₃, NH3, silanes such as SiH₄, tetraethoxysilane, (TEOS), dichlorodimethylsilane (DDS), methyl trichlorosilane (MTS), trichlorosilyl dichloroborane (TDADB), hexadichloromethylsilyl oxide (HDMSO), AICl₃, TiCl₃ or mixtures thereof can be used.

These precursors are used in CVD processes mostly in low concentrations of approximately 0.5 to 15% by vol. in mixture with an inert gas such as e.g. nitrogen, argon or such like. The addition of hydrogen to corresponding deposition gas mixtures is also possible. At temperatures between 500 and 2000° C., preferably 500 to 1500° C. and particularly preferably 700 to 1300° C., the above-mentioned compounds split off hydrocarbon fragments and/or carbon or ceramic precursors which deposit themselves in an essentially evenly distributed manner in the pore system of the pyrolysed coating, modify the pore structure therein and thus lead to an essentially homogeneous pore size and pore distribution.

By means of CVD methods, pores in the carbon-containing layer on the implant can be reduced in size in a controlled manner until the pores are completely closed/sealed off. As a result, the sorptive properties as well as the mechanical properties of the implant surface can be adjusted in a tailor made manner.

By CVD of silanes or siloxanes, if necessary in mixture with hydrocarbons, the carbon-containing implant coatings can be modified e.g. in an oxidation resistant manner by the formation of carbide or oxycarbides.

In preferred embodiments, the implants coated according to the invention can additionally be coated and/or modified by the sputter process. For this purpose, carbon, silicon or metals and/or metal compounds can be applied from suitable sputter targets by methods known as such. Examples of these are Ti, Zr, Ta, W, Mo, Cr, Cu which can be introduced as dusts into the carbon-containing layers, the corresponding carbides being formed as a rule.

Moreover, the surface properties of the coated implant can be modified by ion implantation. By implanting nitrogen, it is thus possible to form nitride phases, carbonitride phases or oxynitride phases with incorporated transition metals thus substantially improving the chemical resistance and the mechanical resistance of the carbon-containing implant coatings. The ion implantation of carbon can be used to increase the mechanical strength of the coatings and for post-compacting porous layers.

Moreover, it is preferred in the case of certain embodiments to fluoridate implant coatings produced according to the invention in order to make surface-coated implants such as e.g. stents or orthopaedic implants, for example, utilisable for the absorption of lipophilic active principles.

In certain embodiments, it may be advantageous to at least partially coat the coated implantable device with at least one additional layer of biodegradable and/or resorbable polymers such as collagen, albumin, gelatine, hyaluronic acid, starch, celluloses such as methylcellulose, hydroxypropyl cellulose, hydroxypropyl methylcellulose, carboxymethylcellulose phthalate; casein, dextrans, polysaccharides, fibrinogen, poly(D,L-lactides), poly(D,L-lactide coglycolides), poly(glycolides), poly(hydroxybutylates), poly(alkyl carbonates), poly(orthoesters), polyesters, poly(hydroxyvaleric acid), polydioxanones, poly(ethylene terephthalates), poly(maleate acid), poly(tartronic acid), polyanhydrides, polyphosphazenes, poly(amino acids) and their copolymers or non-biodegradable and/or resorbable polymers. Anionic, cationic or amphoteric coatings, in particular, such as e.g. alginate, carrageenan, carboxymethylcellulose; chitosan, poly-L-lysine; and/or phosphoryl choline are preferred.

Insofar as required, it is possible in particularly preferred embodiments for the coated implant to be subjected to further chemical or physical surface modifications after carbonising and/or after-treatment steps which may, if necessary, have taken place. Purification steps for the removable of possible residues and impurities may be provided for. For this purpose, acids, in particular oxidising acids or solvents can be used, boiling in acids or solvents being preferred.

Before use for medical purposes, the implants coated according to the invention can be sterilised by the usual methods, e.g. by autoclaving, ethylene oxide sterilisation or gamma irradiation.

Pyrolytic carbon itself produced according to the invention from polymer films is usually a highly biocompatible material which can be used in medical applications such as the external coating of implants. The biocompatibility of the implants coated according to the invention can also be influenced and/or modified in a controlled manner by the incorporation of additives, fillers, proteins or functional materials and/or medicaments into the polymer films before or after carbonising, as mentioned above. In this way, rejection phenomena in the body can be reduced or eliminated altogether in the case of implants produced according to the invention.

In particularly preferred embodiments, carbon-coated medical implants produced according to the invention can be used by the controlled adjustment of the porosity of the carbon layer applied for the controlled release of active principles from the substrate into the external surroundings. Preferred coatings are porous, in particular nanoporous. In this case, it is possible, for example, to use medical implants, in particular also stents, as carriers of medicines with a depot effect, it being possible to utilise the carbon-based coating of the implant as a release-regulating membrane.

It is also possible to apply medicines onto the biocompatible coatings. This is useful in particular in those cases where active principles cannot be applied directly into or onto the implant such as e.g. in the case of metals.

Moreover, the coatings produced according to the invention can be loaded in a further process step with medicines and/or medicaments or with labels, contrast agents for localising coated implants in the body, e.g. also with therapeutic or diagnostic quantities of sources of radioactive radiation. For the latter, the coatings based on carbon according to the invention are particularly suitable since, in contrast to polymer layers, they are not negatively affected or attacked by radioactive radiation.

In the medical area, the implants coated according to the invention have proved to be particularly stable in the long term since the carbon-based coatings can be adjusted regarding their elasticity and flexibility, apart from exhibiting a high level of strength, in such a way that they are able to follow the movements of the implant, in particular in the case of joints subject to a high level of stress, without the danger arising that cracks may form or the layer delaminates.

The porosity of coatings applied according to the invention onto implants can be adjusted in particular also by after-treatment with oxidising agents, e.g. activating at elevated temperatures in oxygen or oxygen-containing atmospheres or the use of strongly oxidising acids such as concentrated nitric acid and such like, in such a way that the carbon-containing surface on the implant allows and promotes the ingrowth of body tissue. Suitable layers for this purpose are macroporous with pore sizes of 0,1 μm to 1000 μm, preferably 1 μm to 400 μm. The appropriate porosity can also be influenced by a corresponding pre-structurisation of the implant or the polymer film. Suitable measures in this respect are e.g. embossing, punching, perforating, foaming of the polymer film.

In preferred embodiments, the implants coated in a biocompatible manner according to the invention can be loaded with active principles, including microorganisms or living cells. Loading with active principles can take place in or on the carbon-containing coating by means of suitable sorptive methods such as adsorption, absorption, physisorption, chemisorption, in the most simple case by impregnating the carbon-containing coating with solutions of the active principle, dispersions of the active principle or suspensions of the active principle in suitable solvents. Covalent or non-covalent bonding of active principles into or onto the carbon-containing coating can also be a preferred option in this case, depending on the active principle used and its chemical properties.

In porous, carbon-containing coatings, active principles can be occluded in pores.

Loading with active principle can be temporary, i.e. the active principle can be liberated after implanting of the medical device or the active principle is permanently immobilised in or on the carbon-containing layer. In this way, medical implants containing active principle can be produced with static, dynamic or combined static and dynamic active principle loadings. In this way, multifunctional coatings based on the carbon-containing layers produced according to the invention are obtained.

In the case of static loadings with active principles, the active principles are immobilised essentially permanently on or in the coating. Active principles that can be used for this purpose are inorganic substances, e.g. hydroxyl apatite (HAP), fluoroapatite, tricalcium phosphate (TCP), zinc; and/or organic substances such as peptides, proteins, carbohydrates such as monosaccharides, oligosaccharides and polysaccharides, lipids, phospholipids, steroids, lipoproteins, glycoproteins, glycolipids, proteoglycanes, DNA, RNA, signal peptides or antibodies and/or antibody fragments, bioresorbable polymers, e.g. polylactonic acid, chitosan and pharmacologically active substances or mixtures of substances, combinations of these and such like.

In the case of dynamic loading with active principles, the release of the applied active principles following implantation of the medical device in the body is provided for. In this way, the coated implants can be used for therapeutic purposes, the active principles applied onto the implant being liberated locally and successively at the site of use of the implant. Active principles that can be used in dynamic loadings of active principles for the release of active principles consist, for example, of hydroxyl apatite (HAP), fluoroapatite, tricalcium phosphate (TCP), zinc; and/or organic substances such as peptides, proteins, carbohydrates such as monosaccharides, oligosaccharides and polysaccharides, lipids, phospholipids, steroids, lipoproteins, glycoproteins, glycolipids, proteoglycanes, DNA, RNA, signal peptides or antibodies and/or antibody fragments, bioresorbable polymers, e.g. polylactonic acid, chitosan and the like as well as pharmacologically active substances and mixtures of substances.

Suitable pharmacologically effective substances or mixtures of substances for static and/or dynamic loading of implantable medical devices coated according to the invention comprise active principles or combinations of active principles which are selected from heparin, synthetic heparin analogues (e.g. fondaparinux), hirudin, antithrombin III, drotrecogin alpha; fibrinolytics such as alteplase, plasmin, lysokinase, factor xiia, prourokinase, urokinase, anistreplase, streptokinase; thrombocyte aggregation inhibitors such as acetyl salicylic acid, ticlopidine, clopidogrel, abciximab, dextrans; corticosteroids such as aldlometasones, amcinonides, augmented betamethasones, beclomethasones, betamethasones, budesonides, cortisones, clobetasol, clocortolones, desonides, desoximetasones, dexamethasones, flucinolones, fluocinonides, flurandrenolides, flunisolides, fluticasones, halcinonides, halobetasol, hydrocortisones, methylprednisolones, mometasones, prednicarbates, prednisones, prednisolones, triamcinolones; so-called non-steroidal anti-inflammatory drugs such as diclofenac, diflunisal, etodolac, fenoprofen, flurbiprofen, ibuprofen, indomethacin, ketoprofen, ketorolac, meclofenamates, mefenamic acid, meloxicam, nabumetones, naproxen, oxaprozin, piroxicam, salsalate, sulindac, tolmetin, celecoxib, rofecoxib; cytostatics such as alkaloids and podophyllum toxins such as vinblastin, vincristin; alkylants such as nitrosoureas, nitrogen lost analogues; cytotoxic antibiotics such as daunorubicin, doxorubicin and other anthracyclins and related substances, bleomycin, mitomycin; antimetabolites such as folic acid analogues, purine analogues or purimidine analogues; paclitaxel, docetaxel, sirolimus; platinum compounds such as carboplatinum, cisplatinum or oxaliplatinum; amsacrin, irinotecan, imatinib, topotecan, interferon-alpha 2a, interferon-alpha 2b, hydroxycarbamide, miltefosin, pentostatin, porfimer, aldesleukin, bexarotene, tretinoin; antiandrogens, and antiestrogens; antiarrythmics, in particular antiarrhythmics of class I such as antiarrhythmics of the quinidine type, e.g. quinidine, dysopyramide, ajmaline, prajmalium bitartrate, detajmium bitartrate; antiarrhythmics of the lidocain type, e.g. lidocain, mexiletin, phenyloin, tocainid; antiarrhythmics of class I C, e.g. propafenone, flecainide (acetate); antiarrhythmics of class II, betareceptor blockers such as metoprolol, esmolol, propranolol, metoprolol, atenolol, oxprenolol; antiarrhythmics of class III such as amiodaron, sotalol; antiarrhythmics of class IV such as diltiazem, verapamil, gallopamil; other antiarrhythmics such as adenosine, orciprenaline, ipratropium bromide; agents for stimulating angiogenesis in the myocardium such as vascular endothelial growth factor (VEGF), basic fibroblast growth factor (bFGF), non-viral DNA, viral DNA, endothelial growth factors: FGF-1, FGF-2, VEGF, TGF; antibodies, monoclonal antibodies, anticalins; stem cells, endothelial progenitor cells (EPC); digitalis glycosides such as acetyl digoxin/methyldigoxin, digitoxin, digoxin; heart glycosides such as ouabain, proscillaridin; antihypertonics such as centrally effective antiadrenergic substances, e.g. methyldopa, imidazoline receptor agonists; calcium channel blockers of the dihydropyridine type such as nifedipine, nitrendipine; ACE inhibitors: quinaprilate, cilazapril, moexipril, trandolapril, spirapril, imidapril, trandolapril; angiotensin-II-antagonists: candesartancilexetil, valsartan, telmisartan, olmesartan medoxomil, eprosartan; peripherally effective alpha-receptor blockers such as prazosin, urapidil, doxazosin, bunazosin, terazosin, indoramin; vasodilators such as dihydralazine, diisopropyl amine dichloroacetate, minoxidil, nitropiusside-sodium; other antihypertonics such as indapamide, codergocrin mesilate, dihydroergotoxin methane sulphonate, cicletanin, bosentan, fludrocortisone; phosphodiesterase inhibitors such as milrinone, enoximone and antihypotonics such as in particular adrenergics and dopaminergic substances such as dobutamine, epinephrine, etilefrine, norfenefrine, norepinephrine, oxilofrine, dopamine, midodrine, pholedrine, amezinium methyl; and partial adrenoceptor agonists such as dihydroergotamine; fibronectin, polylysines, ethylene vinyl acetates, inflammatory cytokines such as: TGFβ, PDGF, VEGF, bFGF, TNFα, NGF, GM-CSF, IGF-a, IL-1, IL-8, IL-6, growth hormones; as wll as adhesive substances such as cyanoacrylates, beryllium, silica; and growth factors such as erythropoietin, hormones such as corticotropins, gonadotropins, sonlatropin, thyrotrophin, desmopressin, terlipressin, oxytocin, cetrorelix, corticorelin, leuprorelin, triptorelin, gonadorelin, ganirelix, buserelin, nafarelin, goserelin, as well as regulatory peptides such as somatostatin, octreotide; bone and cartilage stimulating peptides, bone morphogenetic proteins (BMPs), in particular recombinant BMPs such as e.g. recombinant human BMP-2 (rhBMP-2)), bisphosphonates (e.g. risedronates, pamidronates, ibandronates, zoledronic acid, clodronic acid, etidronic acid, alendronic acid, tiludronic acid), fluorides such as disodium fluorophosphate, sodium fluoride; calcitonin, dihydrotachystyrene; growth factors and cytokines such as epidermal growth factors (EGF), Platelet derived growth factor (PDGF), Fibroblast Growth Factors (FGFs), Transforming Growth Factors-b TGFs-b), Transforming Growth Factor-a (TGF-a), Erythropoietin (Epo), Insulin-Like Growth Factor-I (IGF-I), Insulin-Like Growth Factor-II (IGF-II), Interleukin-1 (IL-1), Interleukin-2 (IL-2), Interleukin-6 (IL-6), Interleukin-8 (IL-8), Tumour Necrosis Factor-a (TNF-a), Tumour Necrosis Factor-b (TNF-b), Interferon-g (INF-g), Colony Stimulating Factors (CSFs); monocyte chemotactic protein, fibroblast stimulating factor 1, histamine, fibrin or fibrinogen, endothelin-1, angiotensin ii, collagens, bromocriptin, methylsergide, methotrexate, carbontetrachloride, thioacetamide and ethanol; also silver (ions), titanium dioxide, antibiotics and antiinfectives such as in particular β-lactam antibiotics, e.g. β-lactamase-sensitive penicillins such as benzyl penicillins (penicillin G), phenoxymethylpenicillin (penicillin V); β-lactamase-resistant penicillins such as aminopenicillins such as amoxicillin, ampicillin, bacampicillin; acylaminopenicillins such as mezlocillin, piperacillin; carboxypenicillines, cephalosporins such as cefazolin, cefuroxim, cefoxitin, cefotiam, cefaclor, cefadroxil, cefalexin, loracarbef, cefixim, cefuroximaxetil, ceftibuten, cefpodoximproxetil, cefpodoximproxetil; aztreonam, ertapenem, meropenem; β-lactamase inhibitors such as sulbactam, sultamicillintosilates; tetracyclines such as doxycycline, minocycline, tetracycline, chlorotetracycline, oxytetracycline; aminoglycosides such as gentamicin, neomycin, streptomycin, tobramycin, amikasin, netilmicin, paromomycin, framycetin, spectinomycin; makrolide antibiotics such as azithromycin, clarithromycin, erythromycin, roxithromycin, spiramycin, josamycin; lincosamides such as clindamycin, lincomycin, gyrase inhibitors such as fluoroquinolones such as ciprofloxacin, ofloxacin, moxifloxacin, norfloxacin, gatifloxacin, enoxacin, fleroxacin, levofloxacin; quinolones such as pipemidic acid; sulphonamides, trimethoprim, sulphadiazin, sulphalene; glycopeptide antibiotics such as vancomycin, teicoplanin; polypeptide antibiotics such as polymyxins such as colistin, polymyxin-b, nitroimidazol derivatives such as metronidazol, tinidazol; aminoquinolones such as chloroquin, mefloquin, hydroxychloroquin; biguanides such as proguanil; quinine alkaloids and diaminopyrimidines such as pyrimethamine; amphenicols such as chloramphenicol; rifabutin, dapsone, fusidinic acid, fosfomycin, nifuratel, telithromycin, fusafungin, fosfomycin, pentamidindiisethionate, rifampicin, taurolidine, atovaquone, linezolid; virostatics such as aciclovir, ganciclovir, famciclovir, foscamet, inosine (dimepranol-4-acetamidobenzoate), valganciclovir, valaciclovir, cidofovir, brivudin; antiretroviral active principles (nucleoside analogous reverse transcriptase inhibitors and derivatives) such as lamivudin, zalcitabin, didanosine, zidovudin, tenofovir, stavudin, abacavir; non-nucleoside analogous reverse transcriptase inhibitors: amprenavir, indinavir, saquinavir, lopinavir, ritonavir, nelfinavir; amantadine, ribavirin, zanamivir, oseltamivir and lamivudine, as well as any desired combination and mixtures thereof.

Particularly preferred embodiments of the present invention which can be produced according to the process of the invention consist of coated vascular endoprostheses (intraluminal endoprostheses) such as stents, coronary stents, intravascular stents, peripheral stents and such like.

These can be coated in a simple and biocompatible manner by the process according to the invention as a result of which the restenoses frequently occurring with conventional stents in the percutaneous transluminal angioplasties, for example, can be prevented.

In preferred embodiments of the invention, it is possible to increase the hydrophilicity of the coating by activating the carbon-containing coating e.g. with air at elevated temperatures, this additionally improving the biocompatibility.

In particularly preferred embodiments, stents provided with a carbon-containing layer according to the process of the invention, in particular coronary stents and peripheral stents, are loaded with pharmacologically effective substances or mixtures of substances. It is, for example, possible to equip the stent surfaces with the following active principles for the local suppression of cell adhesion, thrombocyte aggregation, complement activation and/or inflammatory tissue reactions or cell proliferation:

Heparin, synthetic heparin analogues (e.g. fondaparinux), hirudin, antithrombin III, drotrecogin alpha, fibrinolytics (alteplase, plasmin, lysokinases, factor xiia, prourokinase, urokinase, anistreplase, streptokinase), thrombocyte aggregation inhibitors (acetyl salicylic acid, ticlopidines, clopidogrel, abciximab, dextrans), corticosteroids (alclometasones, amcinonides, augmented betamethasones, beclomethasones, betamethasones, budesonides, cortisones, clobetasol, clocortolones, desonides, desoximetasones, dexamethasones, flucinolones, fluocinonides, flurandrenolides, flunisolides, fluticasones, halcinonides, halobetasol, hydrocortisones, methyl prednisolones, mometasones, prednicarbates, prednisones, prednisolones, triamcinolones), so-called non-steroidal anti-inflammatory drugs (diclofenac, diflunisal, etodolac, fenoprofen, flurbiprofen, ibuprofen, indomethacin, ketoprofen, ketorolac, meclofenamate, mefenamic acid, meloxicam, nabumetones, naproxen, oxaprozin, piroxicam, salsalates, sulindac, tolmetin, celecoxib, rofecoxib), cytostatics (alkaloids and podophyllum toxins such as vinblastin, vincristin; alkylants such as nitrosoureas, nitrogen lost analogues; cytotoxic antibiotics such as daunorubicin, doxorubicin and other anthracyclines and related substances, bleomycin, mitomycin; antimetabolites such as folic acid analogues, purine analogues or pyrimidine analogues; paclitaxel, docetaxel, sirolimus; platinum compounds such as carboplatinum, cisplatinum or oxaliplatinum; amsacrin, irinotecan, imatinib, topotecan, interferon-alpha 2a, interferon-alpha 2b, hydroxycarbamide, miltefosine, pentostatin, porfimer, aldesleukin, bexaroten, tretinoin; antiandrogens, antiestrogens).

For systemic cardiological effects, the stents coated according to the invention can be loaded with: antiarrythmics, in particular antiarrhythmics of class I (antiarrhythmics of the quinidine type, e.g. quinidine, dysopyramide, ajmaline, prajmalium bitartrate, detajmium bitartrate; antiarrhythmics of the lidocain type: lidocain, mexiletin, phenyloin, tocainid; antiarrhythmics of class I C: propafenone, flecainide (acetate)); antiarrhythmics of class II (betareceptor blockers) (metoprolol, esmolol, propranolol, metoprolol, atenolol, oxprenolol); antiarrhythmics of class III (amiodaron, sotalol), antiarrhythmics of class IV (diltiazem, verapamil, gallopamil), other antiarrhythmics such as adenosine, orciprenaline, ipratropium bromide; agents for stimulating the angiogenesis the myocardium: vascular endothelial growth factor (VEGF), basic fibroblast growth factor (bFGF), non-viral DNA, viral DNA, endothelial growth factors: FGF-1, FGF-2, VEGF, TGF; antibodies, monoclonal antibodies, anticalins; stem cells, endothelial progenitor cells (EPC). Further cardiacs are: digitalis glycosides (acetyl digoxin/methyldigoxin, digitoxin, digoxin), further heart glycosides (ouabain, proscillaridin). Also antihypertonics (centrally effective antiadrenergic substances; methyldopa, imidazoline receptor agonists; calcium channel blockers of the dihydropyridine type such as nifedipine, nitrendipine; ACE inhibitors: quinaprilate, cilazapril, moexipril, trandolapril, spirapril, imidapril, trandolapril; angiotensin-II-antagonists: candesartancilexetil, valsartan, telmisartan, olmesartan medoxomil, eprosartan; peripherally effective alpha-receptor blockers; prazosin, urapidil, doxazosin, bunazosin, terazosin, indoramin; vasodilators: dihydralazine, diisopropyl amine dichloroacetate, minoxidil, nitroprusside-sodium), other antihypertonics such as indapamide, codergocrin mesilate, dihydroergotoxin methane sulphonate, cicletanin, bosentan. Also phosphodiesterase inhibitors (milrinone, enoximon) and antihypotonics, here in particular adrenergics and dopaminergic substances (dobutamine, epinephrine, etilefrine, norfenefrine, norepinephrine, oxilofrine, dopamine, midodrine, pholedrine, amezinium methyl), partial adrenoceptor agonists (dihydroergotamine), finally other antihypotonics such as fludrocortisone.

To increase the tissue adhesion, in particular in the case of peripheral stents, components of the extracellular matrix, fibronectin, polylysins, ethylene vinyl acetate, inflammatory cytokines such as: TGFβ, PDGF, VEGF, bFGF, TNFα, NGF, GM-CSF, IGF-a, IL-1, IL-8, IL-6, growth hormones; and adhesive substances such as cyanoacrylates, beryllium or silica can be used:

Further substances suitable for this purpose having a systemic and/or local effect are growth factors, erythropoietin.

The use of hormones can also be provided for in the stent coatings such as for example corticotropins, gonadotropins, somatropin, thyrotrophin, desmopressin, terlipressin, oxytocin, cetrorelix, corticorelin, leuprorelin, triptorelin, gonadorelin, ganirelix, buserelin, nafarelin, goserelin, as well as regulatory peptides such as somatostatin and/or octreotide.

In the case of surgical and orthopaedic implants, it may be advantageous to equip the implants with one or several carbon-containing layers which are macroporous. Suitable pore sizes are in the region of 0.1 to 1000 μm, preferably 1 to 400 μm, in order to enhance an improved integration of the implants by ingrowth into the surrounding cell tissue or bone tissue.

For orthopaedic and non-orthopaedic implants and heart valves or artificial heart parts coated according to the invention it is, moreover, possible—insofar as these are to be loaded with active principles—to use the same active principles as for the stent applications described above for the local suppression of cell adhesion, thrombocyte aggregation, complement activation and/or inflammatory tissue reaction or cell proliferation.

Moreover, the following active principles can be used to stimulate tissue growth, in particular in the case of orthopaedic implants, for a better implant integration: bone and cartilage stimulating peptides, bone morphogenetic proteins (BMPs), in particular recombinant BMPs (e.g. recombinant human BMP-2 (rhBMP-2)), bisphosphonates (e.g. risedronates, pamidronates, ibandronates, zoledronic acid, clodronic acid, etidronic acid, alendronic acid, tiludronic acid), fluorides (disodium fluorophosphate, sodium fluoride); calcitonin, dihydrotachystyrene). Also, all growth factors and cytokines such as epidermal growth factors (EGF), Platelet-derived growth factor (PDGF), Fibroblast Growth Factors (FGFs), Transforming Growth Factors-b TGFs-b), Transforming Growth Factor-a (TGF-a), Erythropoietin (Epo), Insulin-Like Growth Factor-I (IGF-I), Insulin-Like Growth Factor-II (IGF-II), Interleukin-1 (IL-1), Interleukin-2 (IL-2), Interleukin-6 (IL-6), Interleukin-8 (IL-8), Tumour Necrosis Factor-a (TNF-a), Tumour Necrosis Factor-b (TNF-b), Interferon-g (INF-g), Colony Stimulating Factors (CSFs). Further adhesion and integration promoting substances, besides the above-mentioned inflammatory cytokines are the monocyte chemotactic protein, fibroblast stimulating factor 1, histamine, fibrin or fibrinogen, endothelin-1, angiotensin II, collagens, bromocriptin, methylsergide, methotrexate, carbontetrachloride, thioacetamide, ethanol.

In addition, it is possible to provide implants coated according to the invention, in particular stents and such like, with antibacterial/antiinfective coatings, instead of or in addition to pharmaceuticals, the following substances or mixtures of substances being suitable for use: silver (ions), titanium dioxide, antibiotics and antiinfectives. In particular beta-lactam antibiotics (β-lactam antibiotics: β-lactamase-sensitive penicillin such as benzyl penicillin (penicillin G), phenoxymethyl penicillin (penicillin V); β-lactamase-resistant penicillin such as aminopenicillin such as amoxicillin, ampicillin, bacampicillin; acylaminopenicillins such as mezlocillin, piperacillin; carboxypenicillins, cephalosporins (cefazolin, cefuroxim, cefoxitin, cefotiam, cefaclor, cefadroxil, cefalexin, loracarbef, cefixim, cefuroximaxetil, ceftibuten, cefpodoximproxetil, cefpodoximproxetil) or others such as aztreonam, ertapenem, meropenem. Further antibiotics are β-lactamase inhibitors (sulbactam, sultamicillintosilate), tetracyclines (doxycycline, minocycline, tetracycline, chlorotetracycline, oxytetracycline), aminoglycosides (gentamicin, neomycin, streptomycin, tobramycin, amikacin, netilmicin, paromomycin, framycetin, spectinomycin), makrolide antibiotics (azithromycin, clarithromycin, erythromycin, roxithromycin, spiramycin, josamycin), lincosamides (clindamycin, lincomycin), gyrase inhibitors (fluoroquinolones such as ciprofloxacin, ofloxacin, moxifloxacin, norfloxacin, gatifloxacin, enoxacin, fleroxacin, levofloxacin; other quinolones such as pipemidic acid), sulphonamides and trimethoprim (sulphadiazin, sulphalene, trimethoprim), glycopeptide antibiotics (vancomycin, teicoplanin), polypeptide antibiotics (polymyxins such as colistin, polymyxin-B), nitroimidazol derivatives (metronidazol, tinidazol), aminoquinolones (chloroquin, mefloquin, hydroxychloroquin), biguanides (proguanil), quinine alkaloids and diaminopyrimidines (pyrimethamine), amphenicols (chloramphenicol) and other antibiotics (rifabutin, dapsone, fusidinic acid, fosfomycin, nifuratel, telithromycin, fusafungin, fosfomycin, pentamide indiisethionate, rifampicin, taurolidine, atovaquone, linezolide). Among the virostatics, the following deserve to be mentioned: aciclovir, ganciclovir, famciclovir, foscarnet, inosine (dimepranol-4-acetamidobenzoate), valganciclovir, valaciclovir, cidofovir, brivudin. These include, but are not limited to, also antiretroviral active principles (nucleoside analogous reverse transcriptase inhibitors and derivatives: lamivudin, zalcitabin, didanosin, zidovudin, tenofovir, stavudin, abacavir; non-nucleoside analogous reverse transcriptase inhibitors: amprenavir, indinavir, saquinavir, lopinavir, ritonavir, nelfinavir) and other virostatics such as amantadine, ribavirin, zanamivir, oseltamivir, lamivudin.

In particularly preferred embodiments of the present invention, the carbon-containing layers produced according to the invention can be suitably modified regarding their chemical or physical properties before or after loading with active principles by using further agents e.g. in order to modify the hydrophilicity, hydrophobicity, electrical conductivity, adhesion and other surface properties. Substances suitable for use for this purpose are biodegradable or non-biodegradable polymers such as in the case of the biodegradable ones, for example: collagens, albumin, gelatine, hyaluronic acid, starch, cellulose (methylcellulose, hydroxypropyl cellulose, hydroxypropyl methylcellulose, carboxymethylcellulose phthalate; also casein, dextrans, polysaccharides, fibrinogen, poly(D,L-lactides), poly(D,L-lactide coglycolides), poly(glycolides), poly(hydroxybutylates), poly(alkyl carbonates), poly(orthoesters), polyesters, poly(hydroxyvaleric acid), polydioxanones, poly(ethylene terephthalates), poly(maleate acid), poly(tartronic acid), polyanhydrides, polyphosphazenes, poly(amino acids) and all their copolymers.

The non-biodegradable ones include, but are not limited to,: poly(ethylene vinyl acetate), silicones, acrylic polymers such as polyacrylic acid, polymethylacrylic acid, polyacrylocyanoacrylate; polyethylenes, polypropylenes, polyamides, polyurethanes, poly(ester urethanes), poly(ether urethane), poly(ester ureas), polyethers such as polyethylene oxide, polypropylene oxide, pluronics, polytetramethylene glycol; vinyl polymers such as polyvinylpyrrolidones, poly(vinyl alcohols), poly(vinyl acetate phthalate).

In general, it can be said that polymers with anionic properties (e.g. alginate, carrageenan, carboxymethylcellulose) or cationic properties (e.g. chitosan, poly-L-lysine etc.) or both (phosphoryl choline) can be produced.

To modify the release properties of implants containing active principles and coated according to the invention, it is possible to produce specific pH dependent or temperature-dependent release properties by applying further polymers, for example. pH-sensitive polymers are, for example, poly(acrylic acid) and derivatives, for example: homopolymers such as poly(amino carboxylic acid), poly(acryl acid), poly(methyl acrylic acid) and their copolymers. This also applies to polysaccharides such as cellulose acetate phthalate, hydroxypropylmethylcellulose phthalate, hydroxypropylmethylcellulose succinate, cellulose acetate, trimellitate and chitosan. Thermosensitive polymers are, for example, poly(N-isopropyl acrylamide cosodium acrylate-co-n-N-alkyl acrylamide), poly(N-methyl-N-n-propyl acrylamide), poly(N-methyl-N-isopropyl acrylamide), poly(N-n-propyl methacrylamide), poly(N-isopropyl acrylamide), poly(N,n-diethyl acrylamide), poly(N-isopropyl methacrylamide), poly(N-cyclopropyl acrylamide), poly(N-ethyl acrylamide), poly(N-ethyl methacrylamide), poly(N-methyl-N-ethyl acrylamide), poly(N-cyclopropyl acrylamide). Further polymers with thermogel characteristics are hydroxypropylcellulose, methylcellulose, hydroxypropyl methylcellulose, ethylhydroxyethylcellulose and pluronics such as F-127, L-122, L-92, L-81, L-61.

It is possible, on the one hand, for the active principles to be adsorbed (non-covalently, covalently) in the pores of the carbon-containing layer, their release being controllable primarily by the pore size and pore geometry. Additional modifications of the porous carbon layer by chemical modification (anionic, cationic) make it possible to modify the release e.g. as a function of the pH. A further application consists of the release of carriers containing active principle, i.e. micro-capsules, liposomes, nanocapsules, nanoparticles, micelles, synthetic phospholipids, gas-dispersions, emulsions, microemulsions, nanospheres etc., which are adsorbed into the pores of the carbon layer and then released therapeutically. By additional covalent or non-covalent modification of the carbon layer, the pores can be occluded such that biologically active principles are protected. Suitable for this purpose are the polysaccharides, lipids etc. which have already been mentioned above, but also the above-mentioned polymers.

Regarding the additional coating of the porous carbon-containing layers produced according to the invention, a distinction can consequently be made between physical barriers such as inert biodegradable substances (e.g. poly-L-lysine, fibronectin, chitosan, heparin etc.) and biologically active barriers. The latter may consist of sterically hindered molecules which are bioactivated physiologically and allow the release of active principles and/or their carriers. Examples are enzymes, which mediate the release, activate biologically active substances or bind non-active coatings and lead to the exposure of active principles. All the mechanisms and properties specifically listed here can be used for both the primary carbon layer produced according to the invention and for layers additionally applied thereon.

The implants coated according to the invention can, in particular applications, also be loaded with living cells or microorganisms. These can settle in suitable porous carbon-containing layers, it being possible to then provide the implant thus occupied with a suitable membrane covering which is permeable to nutrients and active principles produced by the cells or microorganisms, but not to the cells themselves.

In this way, it is possible to produce implants, for example, by using the technology according to the invention which implants contain insulin producing cells which, after being implanted, produce and release insulin in the body as a function of the glucose level in the surrounding.

The invention will now be further described by way of the following non-limiting examples.

### EXAMPLES

The following examples serve the purpose of illustrating the principles according to the invention and are not intended to be restrictive. In detail, different implants or implant materials are coated according to the process of the invention and their properties, in particular regarding biocompatibility, are determined.

### Example 1

### Carbon

A carbon material coated according to the invention was produced as follows: A polymer film was applied onto paper having a substance weight of 38 g/m² by coating the paper repeatedly with a commercial epoxidised phenol resin varnish using a doctor blade and drying it at room temperature. Dry weight 125 g/m². The pyrolysis at 800° C. over 48 hours under nitrogen resulting in a shrinkage of 20% and a loss of weight of 57% gives an asymmetrically constructed carbon sheet with the following dimensions: total thickness 50 micrometres, with 10 micrometres of a dense carbon-containing layer according to the invention on an open pore carbon carrier with a thickness of 40 micrometres which was formed in situ from the paper under pyrolysis conditions. The absorption capacity of the coated carbon material amounted to as much as 18 g ethanol/m².

### Example 2

### Glass

Duroplan® glass is subjected to 15 minutes of ultrasonic cleaning in a surfactant-containing water bath, rinsed with distilled water and acetone and dried. This material is coated by immersion coating with a commercial packaging varnish based on phenol resin in an application weight of 2.0*10⁻⁴ g/cm². Following subsequent carbonisation at 800° C. for 48 hours under nitrogen, a loss of weight of the coating to 0.33*10⁻⁴ g/cm² takes place. The previously colourless coating turns a glossy black and is hardly transparent any longer after carbonisation. A test of the coating hardness with a pencil which is drawn over the coated surface at an angle of 45° with a weight of 1 kg does not lead to any optically perceptible damage of the surface up to a hardness of 5H.

### Example 3

### Glass, CVD Coating (Reference Example)

Duroplan® glass is subjected to 15 minutes of ultrasonic cleaning, rinsed with distilled water and acetone and dried. This material is coated by chemical vapour deposition (CVD) with 0.05*10⁻⁴ g/cm² of carbon. For this purpose, benzene having a temperature of 30° C. is brought into contact in a blubberer through a stream of nitrogen for 30 minutes with the glass surface having a temperature of 1000° C. and deposited on the glass surface as a film. The previously colourless glass surface turns glossy grey and is moderately transparent after deposition. A test of the coating hardness with a pencil which is drawn over the coated surface at an angle of 45° with a weight of 1 kg does not lead to any optically perceptible damage of the surface up to a hardness of 6 B.

### Example 4

### Glass Fibre

Duroplan® glass fibres with a diameter of 200 micrometres are subjected to 15 minutes of ultrasonic cleaning, rinsed with distilled water and acetone and dried. This material is coated by immersion coating with a commercial packaging varnish in an application weight of 2.0*10⁻⁴ g/cm². Following subsequent pyrolysis with carbonisation at 800° C. for 48 hours, a loss of weight of the coating to 0.33*10⁻⁴ g/cm² takes place. The previously colourless coating turns a glossy black and is hardly transparent any longer after carbonisation. A test of the adhesion of the coating by bending in a radius of 180° does not result in any detachment, i.e. optically detectable damage to the surface.

### Example 5

### Stainless Steel

Stainless steel 1.4301 in the form of a 0.1 mm foil (Goodfellow) is subjected to 15 minutes of ultrasonic cleaning, rinsed with distilled water and acetone and dried.

This material is coated by immersion coating with a commercial packaging varnish in an application weight of 2.0*10⁻⁴ g/cm². Following subsequent pyrolysis with carbonisation at 800° C. for 48 hours under nitrogen, a loss of weight of the coating to 0.49*10⁻⁴ g/cm² takes place. The previously colourless coating turns a mat black after carbonisation. A test of the coating hardness with a pencil which is drawn over the coated surface at an angle of 45° with a weight of 1 kg does not lead to any optically perceptible damage of the surface up to a hardness of 4 B.

An adhesive tape peel test during which a strip of Tesa® tape at least 3 cm in length is glued to the surface using the thumb for 60 seconds and subsequently peeled off again from the surface at an angle of 90° results in hardly any adhesions.

### Example 6

### Stainless steel, CVD Coating (Reference Example)

Stainless steel 1.4301 as an 0.1 mm foil (Goodfellow) is subjected to 15 minutes ultrasonic cleaning, rinsed with distilled water and acetone and dried. This material is coated by chemical vapour deposition (CVD) with 0.20*10⁻⁴ g/cm². For this purpose, benzene having a temperature of 30° C. is brought into contact in a blubberer through a stream of nitrogen for 30 minutes with the metal surface having a temperature of 1000° C., decomposed at the high temperatures and deposited on the metal surface as a film. The previously metallic surface turns a glossy black after deposition. A test of the coating hardness with a pencil which is drawn over the coated surface at an angle of 45° and with a weight of 1 kg does not lead to any optically perceptible damage of the surface up to a hardness of 4 B.

A Tesa adhesive tape peel test during which a strip of Tesa® tape at least 3 cm in length is glued to the surface using the thumb for 60 seconds and subsequently peeled off again from the surface at an angle of 90° results in clearly visible grey adhesions.

### Example 7

### Titanium

Titanium 99.6% as an 0.1 mm foil (Goodfellow) is subjected to 15 minutes of ultrasonic cleaning, rinsed with distilled water and acetone and dried. This material is coated by immersion coating with a commercial packaging varnish with 2.2*10⁻⁴ g/cm². Following subsequent pyrolysis with carbonication at 800° C. for 48 hours under nitrogen, a loss of weight of the coating to 0.73*10⁻⁴ g/cm² takes place. The previously colourless coating turns a mat glossy greyish-black. A test of the coating hardness with a pencil which is drawn over the coated surface at an angle of 45° with a weight of 1 kg does not lead to any optical damage of the surface up to a hardness of 8 H. With a paperclip it is also not possible to scratch the coating. A peel test during which a strip of Tesa® tape at least 3 cm in length is glued to the surface using the thumb for 60 seconds and subsequently peeled off again from the surface at an angle of 90° does not result in any adhesions.

### Example 8

### Titan, Refined with CVD

Titanium 99.6% as an 0.1 mm sheet (Goodfellow) is subjected to 15 minutes of ultrasonic cleaning, rinsed with distilled water and acetone and dried. This material is coated by immersion coating with a commercial packaging varnish with 2.2*10⁻⁴ g/cm². Following subsequent pyrolysis with carbonisation at 800° C. for 48 hours under nitrogen, a loss of weight of the coating to 0.73*10⁻⁴ g/cm² takes place. This material is coated further by chemical vapour deposition (CVD) with 0.10*10⁻⁴ g/cm² of carbon. For this purpose, benzene having a temperature of 30° C. is brought into contact in a blubberer through a stream of nitrogen for 30 minutes with the coated metal surface having a temperature of 1000° C., decomposed and deposited on the surface as a film. The previously metallic surface turns a glossy black after the deposition. After cooling to 400° C., the surface is oxidised by passing air over it for a period of 3 hours. A test of the coating hardness with a pencil which is drawn over the coated surface at an angle of 45° with a weight of lkg does not lead to any optically perceptible damage of the surface up to a hardness of 8H.

A peel test during which a strip of Tesa® adhesive tape at least 3 cm in length is glued to the surface using the thumb for 60 seconds and subsequently peeled off again from the surface at an angle of 90° results in grey adhesions.

### Example 9

The titanium surfaces are tested for their biocompatibility in the in vitro Petri dish model using the usual test methods. For this purpose, pieces 16 cm² in size are punched out from the coated materials of examples 2, 7 and 8 and incubated with blood at 37° C., 5% CO₂ for 3 h. For comparison, surfaces of the uncoated materials titanium and glass with the same size were examined. The experiments are carried out with n=3 donors and three sample bodies were measured per surface. The samples are correspondingly prepared and the different parameters (blood platelets, TAT (thrombin-antithrombin complex) and C5a activation) were determined.

The measured value are tested against a blank value as control corresponding to an almost ideal extremely optimistic biocompatibility and two commercially available dialysis membranes (Cuprophan® and Hemophan®) in order to obtain a reference standard. The results are summarised in Table I.

TABLE I
Biocompatibility test

| | Blood | | |
|---|---|---|---|
| | platelet | | |
| | count | TAT | C5a |
| Material | (%) | (ng/ml) | (ng/ml) |
| 1. Blank value | 86.6 | 3.1 | 3.1 |
| 2. Cuprophan roll 05/3126-42 | 64.8 | 40.2 | 70.4 |
| 3. Hemophan type 80 MC 81-512461 | 71.0 | 32.9 | 29.8 |
| 4. Titanium 99.6%, coated, from example 7 | 73.3 | 194.3 | 3.9 |
| 5. Titanium 99.6%, refined, from example 8 | 67.0 | 11.1 | 10.8 |
| 6. Titanium 99.6%, control | 59.6 | >1200.0 | 14.5 |
| 7. Duroplan glass, coated, from example 2 | 73.7 | 137.1 | 11.4 |
| 8. Duroplan glass control | 49.1 | >1233.3 | 25.5 |

The results show a partially substantial improvement in the biocompatibility of the examples according to the invention both in comparison with the dialysis membranes and in comparison with the uncoated samples.

### Example 10

### Cell Growth Test

The coated titanium surface from example 8 and the amorphous carbon from example 1 were examined further for the cell growth of mouse L929 fibroplasts. An uncoated titanium surface was used for comparison. For this purpose, 3×10⁴ cells per sample body were applied onto the previously steam sterilised samples and incubated for 4 days under optimum conditions. Subsequently, the cells were harvested and the cell count was determined automatically per 4 ml of medium. Each sample was measured twice and the average value taken. The results are indicated in Table II.

TABLE II
Cell growth on coated titanium

| Sample material | Cell count per 4 ml |
|---|---|
| Carbon according to example 1 | 6.6 |
| Titanium 99.6%, control | 4.9 |
| Titanium, refined, from example 8 | 7.8 |

These experiments show in an impressive manner the biocompatibility and the cell growth promoting effect of the surfaces coated according to the invention, in particular in the case of the comparison of the two titanium surfaces.

### Example 11

### Coated Stent

A commercially available metal stent from Baun Melsungen AG, type Coroflex 2.5×19 mm, is subjected to 15 minutes of ultrasonic cleaning in a surfactant-containing water bath, rinsed with distilled water and acetone and dried. This material is coated by immersion coating with a commercial packaging varnish based on phenol resin/melamine resin with 2.0*10⁻⁴ g/cm². Following subsequent pyrolysis with carbonisation at 800° C. for 48 hours under nitrogen, a loss of weight of the coating to 0.49*10⁻⁴ g/cm² takes place. The previously highly glossy metallic surface turns a matt black. For a test of the adhesion of the coating by expansion of the stent under 6 bar to a nominal size of 2.5 mm, the coated stent was expanded with a balloon catheter. The subsequent optical assessment under the lens of a microscope did not show any detachment of the homogeneous coating from the metal surface. The absorption capacity of this porous layer amounted to as much as 0.005 g of ethanol.

### Example 12

### Coated Carbostent

A commercially available carbon-coated metal stent from Sorin Biomedica, type Radix Carbostent 5×12 mm, is subjected to 15 minutes of ultrasonic cleaning, rinsed with distilled water and acetone and dried. This material is coated by immersion coating with a commercial packaging varnish based on phenol resin/melamine resin in an application weight of 2.0*10⁻⁴ g/cm². Following subsequent pyrolysis with carbonisation at 800° C. for 48 hours under nitrogen, a loss of weight of the coating to 0.49*10⁻⁴ g/cm² takes place. The previously black surface turns a matt black after carbonisation. For a test of the adhesion of the coating, by expansion of the stent under 6 bar to a nominal size of 5 mm, the coated stent was expanded. The subsequent optical assessment under the lens of a microscope did not show any detachment of the homogeneous coating from the metal surface. The absorption capacity of this porous layer amounted to as much as 0.005 g of ethanol.

### Example 13

### Activation

The coated stent from example 12 is activated for 8 hours by activation with air at 400° C. During this process, the carbon coating is converted into porous carbon. For a test of the adhesion of the coating by expansion of the stent under 6 bar to the nominal size of 5 mm, the coated stent was expanded. The subsequent optical assessment under the lens of a microscope did not show any detachment of the homogeneous coating from the metal surface. The absorption capacity of this now porous layer of the above-mentioned stent model amounted to as much as 0.007 g of ethanol which shows that an additional activation of the carbon-containing layer additionally increases the absorption capacity.

The invention is further described by the following numbered paragraphs:
1. Process for the production of biocompatible coatings on implantable medical devices comprising the following steps:
a) at least partial coating of the medical device with a polymer film by means of a suitable coating and/or application process;
b) heating of the polymer film in an atmosphere which is essentially free from oxygen to temperatures in the region of 200° C. to 2500° C., for the production of a carbon-containing layer on the medical device.

2. Process according to paragraph 1 characterised in that the implantable medical device consists of a material which is selected from carbon, carbon composite material, carbon fibre, ceramic, glass, metals, alloys, bone, stone, minerals or precursors of these or from materials which are converted under carbonisation conditions into their thermostable state.

3. Process according to any one of the preceding paragraphs, characterised in that the implantable medical device is selected from medical or therapeutic implants such as vascular endoprostheses, stents, coronary stents, peripheral stents, orthopaedic implants, bone or joint prostheses, artificial hearts, artificial heart valves, subcutaneous and/or intramuscular implants and such like.

4. Process according to any one of the preceding paragraphs characterised in that the polymer film comprises: homopolymers or copolymers of aliphatic or aromatic polyolefins such as polyethylene, polypropylene, polybutene, polyisobutene, polypentene; polybutadiene; polyvinyls such as polyvinyl chloride or polyvinyl alcohol, poly(meth)acrylic acid, polyacrylocyano acrylate; polyacrylonitril, polyamide, polyester, polyurethane, polystyrene, polytetrafluoroethylene; polymers such as collagen, albumin, gelatine, hyaluronic acid, starch, celluloses such as methylcellulose, hydroxypropyl cellulose, hydroxypropyl methylcellulose, carboxymethylcellulose phthalate; waxes, paraffin waxes, Fischer-Tropsch waxes; casein, dextrans, polysaccharides, fibrinogen, poly(D,L-lactides), poly(D,L-lactide coglycolides), polyglycolides, polyhydroxybutylates, polyalkyl carbonates, polyorthoesters, polyesters, polyhydroxyvaleric acid, polydioxanones, polyethylene terephthalates, polymaleate acid, polytartronic acid, polyanhydrides, polyphosphazenes, polyamino acids; polyethylene vinyl acetate, silicones; poly(ester urethanes), poly(ether urethanes), poly(ester ureas), polyethers such as polyethylene oxide, polypropylene oxide, pluronics, polytetramethylene glycol; polyvinylpyrrolidone, poly(vinyl acetate phthalate) as well as their copolymers, mixtures and combinations of these homopolymers or copolymers.

5. Process according to any one of paragraphs 1 to 3 characterised in that the polymer film comprises alkyd resin, chlorinated rubber, epoxy resin, acrylate resin, phenol resin, amine resin, melamine resin, alkyl phenol resins, epoxidised aromatic resins, oil base, nitro base, polyester, polyurethane, tar, tar-like materials, tar pitch, bitumen, starch, cellulose, waxes, shellac, organic materials of renewable raw materials or combinations thereof.

6. Process according to any one of the preceding paragraphs characterised in that the polymer film is applied as a liquid polymer or polymer solution in a suitable solvent or solvent mixture, if necessary with subsequent drying, or as a polymer solid, if necessary in the form of sheeting or sprayable particles.

7. Process according to paragraph 6 characterised in that the polymer film is applied onto the device by laminating, bonding, immersing, spraying, printing, knife application, spin coating, powder coating or flame spraying.

8. Process according to any one of the preceding paragraphs further comprising the step of depositing carbon and/or silicon by chemical or physical vapour phase deposition (CVD or PVD).

9. Process according to any one of the preceding paragraphs further comprising the sputter application of carbon and/or silicon and/or of metals.

10. Process according to any one of the preceding paragraphs characterised in that the carbon-containing layer is modified by ion implantation.

11. Process according to any one of the preceding paragraphs characterised in that the carbon-containing layer is post-treated with oxidising agents and/or reducing agents, preferably chemically modified by treating the coated device in oxidising acid or alkali.

12. Process according to any one of the preceding paragraphs characterised in that the carbon-containing layer is purified by solvents or solvent mixtures.

13. Process according to any one of the preceding paragraphs characterised in that steps a) and b) are carried out repeatedly in order to obtain a carbon-containing multi-layer coating, preferably with different porosities, by pre-structuring the polymer films or substrates or suitable oxidative treatment of individual layers.

14. Process according to any one of the preceding paragraphs characterised in that several polymer film layers are applied on top of each other in step a).

15. Process according to any one of the preceding paragraphs characterised in that the carbon-containing coated medical device is at least partially coated with at least one additional layer of biodegradable and/or resorbable polymers or non-biodegradable or resorbable polymers.

16. Process according to paragraph 15 characterised in that the biodegradable or resorbable polymers are selected from collagen, albumin, gelatine, hyaluronic acid, starch, celluloses such as methylcellulose, hydroxypropyl cellulose, hydroxypropyl methylcellulose, carboxymethylcellulose phthalate; casein, dextrans, polysaccharides, fibrinogen, poly(D,L-lactides), poly(D,L-lactide coglycolides), polyglycolides, polyhydroxybutylates, polyalkyl carbonates, polyorthoesters, polyesters, polyhydroxyvaleric acid, polydioxanones, polyethylene terephthalates, polymaleate acid, polytartronic acid, polyanhydrides, polyphosphazenes, polyamino acids and their copolymers.

17. Process according to any one of the preceding paragraphs characterised in that the carbon-containing coating on the device is loaded with at least one active principle, with microorganisms or living cells.

18. Process according to paragraph 17 characterised in that the at least one active principle is applied and/or immobilised in pores on or in the coating by adsorption, absorption, physisorption, chemisorption, covalent bonding or non-covalent bonding, electrostatic fixing or occlusion.

19. Process according to any one of paragraphs 17 or 18 characterised in that the at least one active principle is immobilised essentially permanently on or in the coating.

20. Process according to paragraph 19 characterised in that the active principle comprises inorganic substances e.g. hydroxyl apatite (HAP), fluoroapatite, tricalcium phosphate (TCP), zinc; and/or organic substances such as peptides, proteins, carbohydrates such as monosaccharides, oligosaccharides and polysaccharides, lipids, phospholipids, steroids, lipoproteins, glycoproteins, glycolipids, proteoglycanes, DNA, RNA, signal peptides or antibodies and/or antibody fragments, bioresorbable polymers, e.g. polylactonic acid, chitosan as well as pharmacologically active substances or mixtures of substances, combinations of these and such like.

21. Process according to any one of paragraphs 17 or 18 characterised in that the at least one active principle contained in or on the coating is releasable from the coating in a controlled manner.

22. Process according to paragraph 21 characterised in that the active principle releasable in a controlled manner comprises inorganic substances, e.g. hydroxyl apatite (HAP), fluoroapatite, tricalcium phosphate (TCP), zinc; and/or organic substances such as peptides, proteins, carbohydrates such as monosaccharides, oligosaccharides and polysaccharides, lipids, phospholipids, steroids, lipoproteins, glycoproteins, glycolipids, proteoglycanes, DNA, RNA, signal peptides or antibodies and/or antibody fragments, bioresorbable polymers, e.g. polylactonic acid, chitosan and pharmacologically active substances or mixtures of substances.

23. Process according to any one of paragraphs 20 or 22 characterised in that the pharmacologically effective substances are selected from heparin, synthetic heparin analogues (e.g. fondaparinux), hirudin, antithrombin III, drotrecogin alpha; fibrinolytics such as alteplase, plasmin, lysokinase, factor XIIa, prourokinase, urokinase, anistreplase, streptokinase; thrombocyte aggregation inhibitors such as acetyl salicylic acid, ticlopidines, clopidogrel, abciximab, dextrans; corticosteroids such as alclometasones, amcinonides, augmented betamethasones, beclomethasones, betamethasones, budesonides, cortisones, clobetasol, clocortolones, disunites, desoximetasones, dexamethasones, flucinolones, fluocinonides, flurandrenolides, flunisolides, fluticasones, halcinonides, halobetasol, hydrocortisones, methylprednisolones, mometasones, prednicarbates, prednisones, prednisolones, triamcinolones; so-called non-steroidal anti-inflammatory drugs such as diclofenac, diflunisal, etodolac, fenoprofen, flurbiprofen, ibuprofen, indomethacin, ketoprofen, ketorolac, meclofenamates, mefenamic acid, meloxicam, nabumetones, naproxen, oxaprozin, piroxicam, salsalates, sulindac, tolmetin, celecoxib, rofecoxib; cytostatics such as alkaloids and podophyllum toxins such as vinblastin, vincristin; alkylants such as nitrosoureas, nitrogen lost analogues; cytotoxic antibiotics such as daunorubicin, doxorubicin and other anthracyclines and related substances, bleomycin, mitomycin; antimetabolites such as folic acid analogues, purine analogues or purimidine analogues; paclitaxel, docetaxel, sirolimus; platinum compounds such as carboplatinum, cisplatinum or oxaliplatinum; amsacrin, irinotecan, imatinib, topotecan, interferon-alpha 2a, interferon-alpha 2b, hydroxycarbamide, miltefosin, pentostatin, porfimer, aldesleukin, bexarotene, tretinoin; antiandrogens, and antiestrogens; antiarrythmics, in particular antiarrhythmics of class I such as antiarrhythmics of the quinidine type, e.g. quinidine, dysopyramide, ajmaline, prajmalium bitartrate, detajmium bitartrate; antiarrhythmics of the lidocain type, e.g. lidocain, mexiletin, phenyloin, tocainid; antiarrhythmics of class I C, e.g. propafenone, flecainide (acetate); antiarrhythmics of class II, betareceptor blockers such as metoprolol, esmolol, propranolol, metoprolol, atenolol, oxprenolol; antiarrhythmics of class III such as amiodaron, sotalol; antiarrhythmics of class IV such as diltiazem, verapamil, gallopamil; other antiarrhythmics such as adenosine, orciprenaline, ipratropium bromide; agents for stimulating angiogenesis in the myocardium such as vascular endothelial growth factor (VEGF), basic fibroblast growth factor (bFGF), non-viral DNA, viral DNA, endothelial growth factors: FGF-1, FGF-2, VEGF, TGF; antibodies, monoclonal antibodies, anticalins; stem cells, endothelial progenitor cells (EPC); digitalis glycosides such as acetyl digoxin/methyldigoxin, digitoxin, digoxin; heart glycosides such as ouabain, proscillaridin; antihypertonics such as centrally effective antiadrenergic substances, e.g. methyldopa, imidazoline receptor agonists; calcium channel blockers of the dihydropyridine type such as nifedipine, nitrendipine; ACE inhibitors: quinaprilate, cilazapril, moexipril, trandolapril, spirapril, imidapril, trandolapril; angiotensin-II-antagonists: candesartancilexetil, valsartan, telmisartan, olmesartan medoxomil, eprosartan; peripherally effective alpha-receptor blockers such as prazosin, urapidil, doxazosin, bunazosin, terazosin, indoramin; vasodilators such as dihydralazine, diisopropyl amine dichloroacetate, minoxidil, nitroprusside-sodium; other antihypertonics such as indapamide, codergocrin mesilate, dihydroergotoxin methane sulphonate, cicletanin, bosentan, fludrocortisone; phosphodiesterase inhibitors such as milrinone, enoximone and antihypotonics such as in particular adrenergics and dopaminergic substances such as dobutamine, epinephrine, etilefrine, norfenefrine, norepinephrine, oxilofrine, dopamine, midodrine, pholedrine, amezinium methyl; and partial adrenoceptor agonists such as dihydroergotamine; fibronectin, polylysines, ethylene vinyl acetates, inflammatory cytokines such as: TGFβ, PDGF, VEGF, bFGF, TNFα, NGF, GM-CSF, IGF-a, IL-1, IL-8, IL-6, growth hormones; as well as adhesive substances such as cyanoacrylates, beryllium, silica; and growth factors such as erythropoietin, hormones such as corticotropins, gonadotropins, somatropin, thyrotrophin, desmopressin, terlipressin, oxytocin, cetrorelix, corticorelin, leuprorelin, triptorelin, gonadorelin, ganirelix, buserelin, nafarelin, goserelin, as well as regulatory peptides such as somatostatin, octreotide; bone and cartilage stimulating peptides, bone morphogenetic proteins (BMPs), in particular recombinant BMPs such as e.g. recombinant human BMP-2 (rhBMP-2)), bisphosphonates (e.g. risedronates, pamidronates, ibandronates, zoledronic acid, clodronic acid, etidronic acid, alendronic acid, tiludronic acid), fluorides such as disodium fluorophosphate, sodium fluoride; calcitonin, dihydrotachystyrene; growth factors and cytokines such as epidermal growth factors (EGF), Platelet derived growth factor (PDGF), Fibroblast Growth Factors (FGFs), Transforming Growth Factors-b TGFs-b), Transforming Growth Factor-a (TGF-a), Erythropoietin (Epo), Insulin-Like Growth Factor-I (IGF-I), Insulin-Like Growth Factor-II (IGF-II), Interleukin-1 (IL-1), Interleukin-2 (IL-2), Interleukin-6 (IL-6), Interleukin-8 (IL-8), Tumour Necrosis Factor-a (TNF-a), Tumour Necrosis Factor-b (TNF-b), Interferon-g (INF-g), Colony Stimulating Factors (CSFs); monocyte chemotactic protein, fibroblast stimulating factor 1, histamine, fibrin or fibrinogen, endothelin-1, angiotensin II, collagens, bromocriptin, methylsergide, methotrexate, carbontetrachloride, thioacetamide and ethanol; also silver (ions), titanium dioxide, antibiotics and antiinfectives such as in particular β-lactam antibiotics, e.g. β-lactamase-sensitive penicillins such as benzyl penicillins (penicillin G), phenoxymethylpenicillin (penicillin V); β-lactamase-resistant penicillins such as aminopenicillins such as amoxicillin, ampicillin, bacampicillin; acylaminopenicillins such as meziocillin, piperacillin; carboxypenicillins, cephalosporins such as cefazolin, cefuroxim, cefoxitin, cefotiam, cefaclor, cefadroxil, cefalexin, loracarbef, cefixim, cefuroximaxetil, ceftibutene, cefpodoximproxetil, cefpodoximproxetil; aztreonam, ertapenem, meropenem; β-lactamase inhibitors such as sulbactam, sultamicillintosilates; tetracyclines such as doxycycline, minocycline, tetracycline, chlorotetracycline, oxytetracycline; aminoglycosides such as gentamicin, neomycin, streptomycin, tobramycin, amikacin, netilmicin, paromomycin, framycetin, spectinomycin; makrolide antibiotics such as azithromycin, clarithromycin, erythromycin, roxithromycin, spiramycin, josamycin; lincosamides such as clindamycin, lincomycin, gyrase inhibitors such as fluoroquinolones such as ciprofloxacin, ofloxacin, moxifloxacin, norfloxacin, gatifloxacin, enoxacin, fleroxacin, levofloxacin; quinolones such as pipemidic acid; sulphonamides, trimethoprim, sulphadiazin, sulphalene; glycopeptide antibiotics such as vancomycin, teicoplanin; polypeptide antibiotics such as polymyxins such as colistin, polymyxin-B nitroimidazol derivatives such as metronidazol, tinidazol; aminoquinolones such as chloroquin, mefloquin, hydroxychloroquin; biguanides such as proguanil; quinine alkaloids and diaminopyrimidines such as pyrimethamine; amphenicols such as chloramphenicol; rifabutin, dapsone, fusidinic acid, fosfomycin, nifuratel, telithromycin, fusafungin, fosfomycin, pentamidindiisethionate, rifampicin, taurolidine, atovaquone, linezolid; virostatics such as aciclovir, ganciclovir, famciclovir, foscarnet, inosine(dimepranol-4-acetamidobenzoate), valganciclovir, valaciclovir, cidofovir, brivudin; antiretroviral active principles (nucleoside analogous reverse transcriptase inhibitors and derivatives) such as lamivudin, zalcitabin, didanosine, zidovudin, tenofovir, stavudin, abacavir; non-nucleoside analogous reverse transcriptase inhibitors such as amprenavir, indinavir, saquinavir, lopinavir, ritonavir, nelfinavir; amantadine, ribavirin, zanamivir, oseltamivir and lamivudine, as well as any desired combination and mixtures thereof.

24. Process according to any one of paragraphs 20 to 23, characterised in that, the pharmacologically effective substances are incorporated into microcapsules, liposomes, nanocapsules, nanoparticles, micelles, synthetic phospholipids, gas dispersions, emulsions, micro-emulsions, or nanospheres which are reversibly adsorbed and/or absorbed in the pores or on the surface of the carbon-containing layer for later release in the body.

25. Process according to any one of the preceding paragraph characterised in that the implantable medical device consists of a stent consisting of a material selected from the group of stainless steel, platinum-containing radiopaque steel alloys, cobalt alloys, titanium alloys, high-melting alloys based on niobium, tantalum, tungsten and molybdenum, noble metal alloys, nitinol alloys as well as magnesium alloys and mixtures of the above-mentioned substances.

26. Biocompatibly coated implantable medical device comprising a carbon-containing surface coating, producible according to one of the preceding paragraphs.

27. Device according to paragraph 26, consisting of metals such as stainless steel, titanium, tantalum, platinum, nitinol or nickel-titanium alloy; carbon fibres, full carbon material, carbon composite, ceramic, glass or glass fibres.

28. Device according to paragraph 26 or 27, comprising several carbon-containing layers, preferably with different porosities.

29. Device according to any one of paragraphs 26 to 28, additionally comprising a coating of biodegradable and/or resorbably polymers such as collagen, albumin, gelatine, hyaluronic acid, starch, celluloses such as methylcellulose, hydroxypropyl cellulose, hydroxypropyl methylcellulose, carboxymethylcellulose phthalate; waxes, casein, dextrans, polysaccharides, fibrinogen, poly(D,L-lactides), poly(D,L-lactide coglycolides), poly(glycolides), poly(hydroxybutylates), poly(alkyl carbonates), poly(orthoesters), polyesters, poly(hydroxyvaleric acid), polydioxanones, poly(ethylene terephthalates), poly(maleate acid), poly(tartronic acid), polyanhydrides, polyphosphazenes, poly(amino acids) and their copolymers.

30. Device according to any one of paragraphs 26 to 28, additionally comprising a coating of non-biodegradable and/or resorbably polymers such as poly(ethylene vinyl acetate), silicones, acrylic polymers such as polyacrylic acid, polymethylacrylic acid, polyacrylocyanoacrylate; polyethylenes, polypropylenes, polyamides, polyurethanes, poly(ester urethanes), poly(ether urethane), poly(ester ureas), polyethers, poly(ethylene oxide), poly(propylene oxide), pluronics, poly(tetramethylene glycol); vinyl polymers such as polyvinylpyrrolidones, poly(vinyl alcohols)or poly(vinyl acetate phthalate) as well as their copolymers.

31. Device according to any one of paragraphs 26 to 30, further comprising anionic or cationic or amphoteric coatings such as e.g. alginate, carrageenan, carboxymethylcellulose; chitosan, poly-L-lysines; and/or phosphoryl choline.

32. Device according to any one of paragraphs 26 to 31 characterised in that the carbon-containing layer is porous, preferably macroporous, with pore diameters in the region of 0.1 to 100 μm, and particularly preferably nanoporous.

33. Device according to any one of paragraphs 26 to 31 characterised in that the carbon-containing layer is non-porous and/or essentially contains closed pores.

34. Device according to any one of paragraphs 26 to 33, containing one or several active principles as indicated in paragraph 19.

35. Device according to paragraph 34, further comprising a coating influencing the release of the active principles, selected from pH-sensitive and/or temperature-sensitive polymers and/or biologically active barriers such as enzymes.

36. Coated stent according to any one of paragraphs 26 to 35.

37. Coated stent according to paragraph 36 selected from stainless steel, preferably Fe-18Cr-14Ni-2.5Mo (“316LVM” ASTM F138), Fe-21Cr-10Ni-3.5Mn-2.5Mo (ASTM F 1586), Fe-22Cr-13Ni-5Mn (ASTM F 1314), Fe-23Mn-21Cr-1Mo-1N (nickel-free stainless steel); from cobalt alloys, preferably Co-20Cr-15W-10Ni (“L605” ASTM F90), Co-20Cr-35Ni-10Mo (“MP35N” ASTM F 562), Co-20Cr-16Ni-16Fe-7Mo (“Phynox” ASTM F 1058); from titanium alloys are CP titanium (ASTM F 67, grade 1), Ti-6A1-4V (alpha/beta ASTM F 136), Ti-6A1-7Nb (alpha/beta ASTM F1295), Ti-15Mo (beta grade ASTM F2066); from noble metal alloys, in particular iridium-containing alloys such as Pt-10Ir; nitinol alloys such as martensitic, superelastic and cold worked nitinols as well as magnesium alloys such as Mg-3A1-1Z; as well as at least one carbon-containing surface layer.

38. Coated heart valve according to any one of paragraphs 26 to 35.

39. Device according to any one of paragraphs 26 to 35 in the form of an orthopaedic bone prosthesis or joint prosthesis, a bone substitute or a vertebra substitute in the breast or lumbar region of the spine.

40. Device according to any one of paragraphs 26 to 35 in the form of a subcutaneous and/or intramuscular implant for the controlled release of active principle.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A method for the production of biocompatible coatings on implantable medical devices comprising the following steps:
a) at least partially coating of the medical device with a polymer film by means of a suitable coating and/or application process;
b) heating of the polymer film in an atmosphere which is essentially free from oxygen to temperatures in the region of 200° C. to 2500° C., for the production of a carbon-containing layer on the medical device.

2. The method according to claim 1 wherein the implantable medical device consists of a material which is selected from carbon, carbon composite material, carbon fibre, ceramic, glass, metals, alloys, bone, stone, minerals or precursors of these or from materials which are converted under carbonisation conditions into their thermostable state.

3. The method according to claim 1 wherein the implantable medical device is selected from medical or therapeutic implants such as vascular endoprostheses, stents, coronary stents, peripheral stents, orthopaedic implants, bone or joint prostheses, artificial hearts, artificial heart valves, subcutaneous and/or intramuscular implants and such like.

4. The method according to claim 1 wherein the polymer film comprises: homopolymers or copolymers of aliphatic or aromatic polyolefins such as polyethylene, polypropylene, polybutene, polyisobutene, polypentene; polybutadiene; polyvinyls such as polyvinyl chloride or polyvinyl alcohol, poly(meth)acrylic acid, polyacrylocyano acrylate; polyacrylonitril, polyamide, polyester, polyurethane, polystyrene, polytetrafluoroethylene; polymers such as collagen, albumin, gelatine, hyaluronic acid, starch, celluloses such as methylcellulose, hydroxypropyl cellulose, hydroxypropyl methylcellulose, carboxymethylcellulose phthalate; waxes, paraffin waxes, Fischer-Tropsch waxes; casein, dextrans, polysaccharides, fibrinogen, poly(D,L-lactides), poly(D,L-lactide coglycolides), polyglycolides, polyhydroxybutylates, polyalkyl carbonates, polyorthoesters, polyesters, polyhydroxyvaleric acid, polydioxanones, polyethylene terephthalates, polymaleate acid, polytartronic acid, polyanhydrides, polyphosphazenes, polyamino acids; polyethylene vinyl acetate, silicones; poly(ester urethanes), poly(ether urethanes), poly(ester ureas), polyethers such as polyethylene oxide, polypropylene oxide, pluronics, polytetramethylene glycol; polyvinylpyrrolidone, poly(vinyl acetate phthalate) as well as their copolymers, mixtures and combinations of these homopolymers or copolymers.

5. The method according to claim 1 wherein the polymer film comprises alkyd resin, chlorinated rubber, epoxy resin, acrylate resin, phenol resin, amine resin, melamine resin, alkyl phenol resins, epoxidised aromatic resins, oil base, nitro base, polyester, polyurethane, tar, tar-like materials, tar pitch, bitumen, starch, cellulose, waxes, shellac, organic materials of renewable raw materials or combinations thereof.

6. The method according to claim 1 wherein the polymer film is applied as a liquid polymer or polymer solution in a suitable solvent or solvent mixture, if necessary with subsequent drying, or as a polymer solid, if necessary in the form of sheeting or sprayable particles.

7. The method according to claim 6 wherein the polymer film is applied onto the device by laminating, bonding, immersing, spraying, printing, knife application, spin coating, powder coating or flame spraying.

8. The method according to claim 1 wherein further comprising the step of depositing carbon and/or silicon by chemical or physical vapour phase deposition (CVD or PVD).

9. The method according to claim 1 wherein further comprising a sputter application of carbon and/or silicon and/or of metals.

10. The method according to claim 1 wherein the carbon-containing layer is modified by ion implantation.

11. The method according to claim 1 wherein the carbon-containing layer is post-treated with oxidising agents and/or reducing agents, preferably chemically modified by treating the coated device in oxidising acid or alkali.

12. The method according to claim 1 wherein the carbon-containing layer is purified by solvents or solvent mixtures.

13. The method according to claim 1 wherein steps a) and b) are carried out repeatedly in order to obtain a carbon-containing multi-layer coating, preferably with different porosities, by pre-structuring the polymer films or substrates or suitable oxidative treatment of individual layers.

14. The method according to claim 1 wherein several polymer film layers are applied on top of each other in step a).

15. The method according to claim 1 wherein the carbon-containing coated medical device is at least partially coated with at least one additional layer of biodegradable and/or resorbable polymers or non-biodegradable or resorbable polymers.

16. The method according to claim 15 wherein the biodegradable or resorbable polymers are selected from collagen, albumin, gelatine, hyaluronic acid, starch, celluloses such as methylcellulose, hydroxypropyl cellulose, hydroxypropyl methylcellulose, carboxymethylcellulose phthalate; casein, dextrans, polysaccharides, fibrinogen, poly(D,L-lactides), poly(D,L-lactide coglycolides), polyglycolides, polyhydroxybutylates, polyalkyl carbonates, polyorthoesters, polyesters, polyhydroxyvaleric acid, polydioxanones, polyethylene terephthalates, polymaleate acid, polytartronic acid, polyanhydrides, polyphosphazenes, polyamino acids and their copolymers.

17. The method according to claim 1 wherein the carbon-containing coating on the device is loaded with at least one active principle, microorganisms or living cells.

18. The method according to claim 17 wherein the at least one active principle is applied and/or immobilised in pores on or in the coating by adsorption, absorption, physisorption, chemisorption, covalent bonding or non-covalent bonding, electrostatic fixing or occlusion.

19. The method according to claim 17 wherein the at least one active principle is immobilised essentially permanently on or in the coating.

20. The method according to claim 19 wherein the active principle comprises inorganic substances e.g. hydroxyl apatite (HAP), fluoroapatite, tricalcium phosphate (TCP), zinc; and/or organic substances such as peptides, proteins, carbohydrates such as monosaccharides, oligosaccharides and polysaccharides, lipids, phospholipids, steroids, lipoproteins, glycoproteins, glycolipids, proteoglycanes, DNA, RNA, signal peptides or antibodies and/or antibody fragments, bioresorbable polymers, e.g. polylactonic acid, chitosan as well as pharmacologically active substances or mixtures of substances, combinations of these and such like.

21. The method according to claim 17 wherein the at least one active principle contained in or on the coating is releasable from the coating in a controlled manner.

22. The method according to claim 21 wherein the active principle releasable in a controlled manner comprises inorganic substances, e.g. hydroxyl apatite (HAP), fluoroapatite, tricalcium phosphate (TCP), zinc; and/or organic substances such as peptides, proteins, carbohydrates such as monosaccharides, oligosaccharides and polysaccharides, lipids, phospholipids, steroids, lipoproteins, glycoproteins, glycolipids, proteoglycanes, DNA, RNA, signal peptides or antibodies and/or antibody fragments, bioresorbable polymers, e.g. polylactonic acid, chitosan and pharmacologically active substances or mixtures of substances.

23. The method according claim 20 or 21 wherein the pharmacologically active substances are selected from heparin, synthetic heparin analogues (e.g. fondaparinux), hirudin, antithrombin III, drotrecogin alpha; fibrinolytics such as alteplase, plasmin, lysokinase, factor XIIa, prourokinase, urokinase, anistreplase, streptokinase; thrombocyte aggregation inhibitors such as acetyl salicylic acid, ticlopidines, clopidogrel, abciximab, dextrans; corticosteroids such as alclometasones, amcinonides, augmented betamethasones, beclomethasones, betamethasones, budesonides, cortisones, clobetasol, clocortolones, disunites, desoximetasones, dexamethasones, flucinolones, fluocinonides, flurandrenolides, flunisolides, fluticasones, halcinonides, halobetasol, hydrocortisones, methylprednisolones, mometasones, prednicarbates, prednisones, prednisolones, triamcinolones; so-called non-steroidal anti-inflammatory drugs such as diclofenac, diflunisal, etodolac, fenoprofen, flurbiprofen, ibuprofen, indomethacin, ketoprofen, ketorolac, meclofenamates, mefenamic acid, meloxicam, nabumetones, naproxen, oxaprozin, piroxicam, salsalates, sulindac, tolmetin, celecoxib, rofecoxib; cytostatics such as alkaloids and podophyllum toxins such as vinblastin, vincristin; alkylants such as nitrosoureas, nitrogen lost analogues; cytotoxic antibiotics such as daunorubicin, doxorubicin and other anthracyclines and related substances, bleomycin, mitomycin; antimetabolites such as folic acid analogues, purine analogues or purimidine analogues; paclitaxel, docetaxel, sirolimus; platinum compounds such as carboplatinum, cisplatinum or oxaliplatinum; amsacrin, irinotecan, imatinib, topotecan, interferon-alpha 2a, interferon-alpha 2b, hydroxycarbamide, miltefosin, pentostatin, porfimer, aldesleukin, bexarotene, tretinoin; antiandrogens, and antiestrogens; antiarrythmics, in particular antiarrhythmics of class I such as antiarrhythmics of the quinidine type, e.g. quinidine, dysopyramide, ajmaline, prajmalium bitartrate, detajmium bitartrate; antiarrhythmics of the lidocain type, e.g. lidocain, mexiletin, phenyloin, tocainid; antiarrhythmics of class I C, e.g. propafenone, flecainide (acetate); antiarrhythmics of class II, betareceptor blockers such as metoprolol, esmolol, propranolol, metoprolol, atenolol, oxprenolol; antiarrhythmics of class III such as amiodaron, sotalol; antiarrhythmics of class IV such as diltiazem, verapamil, gallopamil; other antiarrhythmics such as adenosine, orciprenaline, ipratropium bromide; agents for stimulating angiogenesis in the myocardium such as vascular endothelial growth factor (VEGF), basic fibroblast growth factor (bFGF), non-viral DNA, viral DNA, endothelial growth factors: FGF-1, FGF-2, VEGF, TGF; antibodies, monoclonal antibodies, anticalins; stem cells, endothelial progenitor cells (EPC); digitalis glycosides such as acetyl digoxin/methyldigoxin, digitoxin, digoxin; heart glycosides such as ouabain, proscillaridin; antihypertonics such as centrally effective antiadrenergic substances, e.g. methyldopa, imidazoline receptor agonists; calcium channel blockers of the dihydropyridine type such as nifedipine, nitrendipine; ACE inhibitors: quinaprilate, cilazapril, moexipril, trandolapril, spirapril, imidapril, trandolapril; angiotensin-II-antagonists: candesartancilexetil, valsartan, telmisartan, olmesartan medoxomil, eprosartan; peripherally effective alpha-receptor blockers such as prazosin, urapidil, doxazosin, bunazosin, terazosin, indoramin; vasodilators such as dihydralazine, diisopropyl amine dichloroacetate, minoxidil, nitroprusside-sodium; other antihypertonics such as indapamide, codergocrin mesilate, dihydroergotoxin methane sulphonate, cicletanin, bosentan, fludrocortisone; phosphodiesterase inhibitors such as milrinone, enoximone and antihypotonics such as in particular adrenergics and dopaminergic substances such as dobutamine, epinephrine, etilefrine, norfenefrine, norepinephrine, oxilofrine, dopamine, midodrine, pholedrine, amezinium methyl; and partial adrenoceptor agonists such as dihydroergotamine; fibronectin, polylysines, ethylene vinyl acetates, inflammatory cytokines such as: TGFβ, PDGF, VEGF, bFGF, TNFα, NGF, GM-CSF, IGF-a, IL-1, IL-8, IL-6, growth hormones; as well as adhesive substances such as cyanoacrylates, beryllium, silica; and growth factors such as erythropoietin, hormones such as corticotropins, gonadotropins, somatropin, thyrotrophin, desmopressin, terlipressin, oxytocin, cetrorelix, corticorelin, leuprorelin, triptorelin, gonadorelin, ganirelix, buserelin, nafarelin, goserelin, as well as regulatory peptides such as somatostatin, octreotide; bone and cartilage stimulating peptides, bone morphogenetic proteins (BMPs), in particular recombinant BMPs such as e.g. recombinant human BMP-2 (rhBMP-2)), bisphosphonates (e.g. risedronates, pamidronates, ibandronates, zoledronic acid, clodronic acid, etidronic acid, alendronic acid, tiludronic acid), fluorides such as disodium fluorophosphate, sodium fluoride; calcitonin, dihydrotachystyrene; growth factors and cytokines such as epidermal growth factors (EGF), Platelet derived growth factor (PDGF), Fibroblast Growth Factors (FGFs), Transforming Growth Factors-b TGFs-b), Transforming Growth Factor-a (TGF-a), Ervthropoietin (Epo), Insulin-Like Growth Factor-I (IGF-I), Insulin-Like Growth Factor-II (IGF-II), Interleukin-1 (IL-1), Interleukin-2 (IL-2), Interleukin-6 (IL-6), Interleukin-8 (IL-8), Tumour Necrosis Factor-a (TNF-a), Tumour Necrosis Factor-b (TNF-b), Interferon-g (INF-g), Colony Stimulating Factors (CSFs); monocyte chemotactic protein, fibroblast stimulating factor 1, histamine, fibrin or fibrinogen, endothelin-1, angiotensin II, collagens, bromocriptin, methylsergide, methotrexate, carbontetrachloride, thioacetamide and ethanol; also silver (ions), titanium dioxide, antibiotics and antiinfectives such as in particular β-lactam antibiotics, e.g. β-lactamase-sensitive penicillins such as benzyl penicillins (penicillin G), phenoxymethylpenicillin (penicillin V); β-lactamase-resistant penicillins such as aminopenicillins such as amoxicillin, ampicillin, bacampicillin; acylaminopenicillins such as meziocillin, piperacillin; carboxypenicillins, cephalosporins such as cefazolin, cefuroxim, cefoxitin, cefotiam, cefaclor, cefadroxil, cefalexin, loracarbef, cefixim, cefuroximaxetil, ceftibutene, cefpodoximproxetil, cefpodoximproxetil; aztreonam, ertapenem, meropenem; β-lactamase inhibitors such as sulbactam, sultamicillintosilates; tetracyclines such as doxycycline, minocycline, tetracycline, chlorotetracycline, oxytetracycline; aminoglycosides such as gentamicin, neomycin, streptomycin, tobramycin, amikacin, netilmicin, paromomycin, framycetin, spectinomycin; makrolide antibiotics such as azithromycin, clarithromycin, erythromycin, roxithromycin, spiramycin, josamycin; lincosamides such as clindamycin, lincomycin, gyrase inhibitors such as fluoroquinolones such as ciprofloxacin, ofloxacin, moxifloxacin, norfloxacin, gatifloxacin, enoxacin, fleroxacin, levofloxacin; quinolones such as pipemidic acid; sulphonamides, trimethoprim, sulphadiazin, sulphalene; glycopeptide antibiotics such as vancomycin, teicoplanin; polypeptide antibiotics such as polymyxins such as colistin, polymyxin-B nitroimidazol derivatives such as metronidazol, tinidazol; aminoquinolones such as chloroquin, mefloquin, hydroxychloroquin; biguanides such as proguanil; quinine alkaloids and diaminopyrimidines such as pyrimethamine; amphenicols such as chloramphenicol; rifabutin, dapsone, fusidinic acid, fosfomycin, nifuratel, telithromycin, fusafungin, fosfomycin, pentamidindiisethionate, rifampicin, taurolidine, atovaquone, linezolid; virostatics such as aciclovir, ganciclovir, famciclovir, foscarnet, inosine(dimepranol-4-acetamidobenzoate), valganciclovir, valaciclovir, cidofovir, brivudin; antiretroviral active principles (nucleoside analogous reverse transcriptase inhibitors and derivatives) such as lamivudin, zalcitabin, didanosine, zidovudin, tenofovir, stavudin, abacavir; non-nucleoside analogous reverse transcriptase inhibitors such as amprenavir, indinavir, saquinavir, lopinavir, ritonavir, nelfinavir; amantadine, ribavirin, zanamivir, oseltamivir and lamivudine, as well as any desired combination and mixtures thereof.

24. The method according to claim 20 or 21, characterised in that, the pharmacologically active substances are incorporated into microcapsules, liposomes, nanocapsules, nanoparticles, micelles, synthetic phospholipids, gas dispersions, emulsions, micro-emulsions, or nanospheres which are reversibly adsorbed and/or absorbed in the pores or on the surface of the carbon-containing layer for later release in the body.

25. The method according to claim 1 wherein the implantable medical device consists of a stent consisting of a material selected from the group of stainless steel, platinum-containing radiopaque steel alloys, cobalt alloys, titanium alloys, high-melting alloys based on niobium, tantalum, tungsten and molybdenum, noble metal alloys, nitinol alloys as well as magnesium alloys and mixtures of the above-mentioned substances.

26. A biocompatibly coated implantable medical device comprising a carbon-containing surface coating, produced according to the method of claim 1.

27. The device according to claim 26, wherein the device further comprises metals such as stainless steel, titanium, tantalum, platinum, nitinol or nickel-titanium alloy; carbon fibres, full carbon material, carbon composite, ceramic, glass or glass fibres.

28. The device according to claim 26, wherein the device further comprises several carbon-containing layers, preferably with different porosities.

29. The device according to claim 26, wherein the device further comprises a coating of biodegradable and/or resorbably polymers such as collagen, albumin, gelatine, hyaluronic acid, starch, celluloses such as methylcellulose, hydroxypropyl cellulose, hydroxypropyl methylcellulose, carboxymethylcellulose phthalate; waxes, casein, dextrans, polysaccharides, fibrinogen, poly(D,L-lactides), poly(D,L-lactide coglycolides), poly(glycolides), poly(hydroxybutylates), poly(alkyl carbonates), poly(orthoesters), polyesters, poly(hydroxyvaleric acid), polydioxanones, poly(ethylene terephthalates), poly(maleate acid), poly(tartronic acid), polyanhydrides, polyphosphazenes, poly(amino acids) and their copolymers.

30. The device according to claim 26, wherein the device further comprises a coating of non-biodegradable and/or resorbably polymers such as poly(ethylene vinyl acetate), silicones, acrylic polymers such as polyacrylic acid, polymethylacrylic acid, polyacrylocyanoacrylate; polyethylenes, polypropylenes, polyamides, polyurethanes, poly(ester urethanes), poly(ether urethane), poly(ester ureas), polyethers, poly(ethylene oxide), poly(propylene oxide), pluronics, poly(tetramethylene glycol); vinyl polymers such as polyvinylpyrrolidones, poly(vinyl alcohols)or poly(vinyl acetate phthalate) as well as their copolymers.

31. The device according to claim 26, wherein the device further comprises anionic or cationic or amphoteric coatings such as alginate, carrageenan, carboxymethylcellulose; chitosan, poly-L-lysines; and/or phosphoryl choline.

32. The device according to claim 26 wherein the carbon-containing surface coating is porous, preferably macroporous, with pore diameters in the region of 0.1 to 1000 μm, and particularly preferably nanoporous.

33. The device according to claim 26 wherein the carbon-containing surface coating is non-porous and/or essentially contains closed pores.

34. The device according to claim 26, wherein the device further comprises one or several active principles comprising inorganic substances e.g. hydroxyl apatite (HAP), fluoroapatite, tricalcium phosphate (TCP), zinc; and/or organic substances such as peptides, proteins, carbohydrates such as monosaccharides, oligosaccharides and polysaccharides, lipids, phospholipids, steroids, lipoproteins, glycoproteins, glycolipids, proteoglycanes, DNA, RNA, signal peptides or antibodies and/or antibody fragments, bioresorbable polymers, e.g. polylactonic acid, chitosan as well as pharmacologically active substances or mixtures of substances, combinations of these and such like.

35. The device according to claim 34, wherein the device further comprises a coating influencing the release of the active principles, selected from pH-sensitive and/or temperature-sensitive polymers and/or biologically active barriers such as enzymes.

36. A coated stent comprising the device of claim 26.

37. The coated stent according to claim 36, wherein the stent comprises stainless steel, preferably Fe-18Cr-14Ni-2.5Mo (“316LVM” ASTM F138), Fe-21Cr-10Ni-3.5Mn-2.5Mo (ASTM F 1586), Fe-22Cr-13Ni-5Mn (ASTM F 1314), Fe-23Mn-21Cr-1Mo-1N (nickel-free stainless steel); from cobalt alloys, preferably Co-20Cr-5W-10Ni (“L605” ASTM F90), Co-20Cr-35Ni-10Mo (“MP35N” ASTM F 562), Co-20Cr-16Ni-16Fe-7Mo (“Phynox” ASTM F 1058); from titanium alloys are CP titanium (ASTM F 67, grade 1), Ti-6A1-4V (alpha/beta ASTM F 136), Ti-6A1-7Nb (alpha/beta ASTM F1295), Ti-15Mo (beta grade ASTM F2066);
from noble metal alloys, in particular iridium-containing alloys such as Pt-10Ir; nitinol alloys such as martensitic, superelastic and cold worked nitinols as well as magnesium alloys such as Mg-3A1-1Z; as well as at least one carbon-containing surface layer.

38. A coated heart valve comprising the device of claim 26.

39. The device according to claim 26 wherein the device is an orthopaedic bone prosthesis or joint prosthesis, a bone substitute or a vertebra substitute in the breast or lumbar region of the spine.

40. The device according to claim 26 wherein the device is a subcutaneous and/or intramuscular implant for the controlled release of active principle.
